(19) United States
(12) Patent Application Publication
(10) Pub. No.: US 2014/0294942 A1
(43) Pub. Date: Oct. 2, 2014

(54) ANTIVIRAL PEPTIDES EFFECTIVE AGAINST HEPATITIS C VIRUS

(71) Applicants: Samuel Wheeler French, Los Angeles, CA (US); Ronik Khachatoorian, Northridge, CA (US); Piotr Pawel Ruchala, Los Angeles, CA (US)

(72) Inventors: Samuel Wheeler French, Los Angeles, CA (US); Ronik Khachatoorian, Northridge, CA (US); Piotr Pawel Ruchala, Los Angeles, CA (US)

(73) Assignee: THE REGENTS OF THE UNIVERSITY OF CALIFORNIA, Oakland, CA (US)

(21) Appl. No.: 14/342,512

(22) PCT Filed: Sep. 6, 2012
(86) PCT No.: PCT/US2012/053936

§ 371 Date: Jun. 2, 2014

Related U.S. Application Data
(60) Provisional application No. 61/531,998, filed on Sep. 7, 2011.

(51) Int. Cl. C07K 14/47; C07K 7/08; C07K 7/64; C07K 7/06

(52) U.S. Cl. CPC C07K 14/47; C07K 7/06; C07K 7/08; C07K 7/64
USPC 424/450; 530/328; 530/327; 530/326; 530/325; 530/324; 530/321; 514/3.7

(57) ABSTRACT

In certain embodiments this invention provides novel antiviral peptide(s) that are effective against positive sense RNA viruses that have an internal ribosome entry site (IRES). The peptide(s) can be used to inhibit propagation of such viruses and thereby provide a effective modality for the treatment of infections such as hepatitis C, and the like.

### CROSS-REFERENCE TO RELATED APPLICATIONS

This application claims benefit of and priority to U.S. Ser. No. 61/531,998, filed Sep. 7, 2011, which is incorporated herein by reference in its entirety for all purposes.

### STATEMENT OF GOVERNMENTAL SUPPORT

This invention was made with Government support of Grant No. DK090794, awarded by the National Institutes of Health. The Government has certain rights in this invention. This work was supported by the U.S. Department of Veterans Affairs, and the Federal Government has certain rights in this invention.

### BACKGROUND

Hepatitis C virus (HCV) infection has a worldwide prevalence of 3% and is the main indication for liver transplantation in developed countries for treatment of cirrhosis (Shepard et al. (2005) The Lancet Infectious Diseases, 5: 558-567). In the United States, HCV is the most common chronic blood borne infection affecting 1.8% of the population and is the major etiologic factor responsible for the recent doubling of hepatocellular carcinoma (HCC) (El-Serag (2002) J. Clin. Gastroenterol., 35: S72-78).

Current HCV therapy consists of pegylated interferon-α (PEG-IFN) and ribavirin which is suboptimal as 70-80% of US patients are infected with genotype 1, with sustained virologic response (SVR) of only 50-56% (Gambarin-Gelwan and Jacobson (2008) J. Viral Hepatitis, 15: 623-633). Generally, therapy of all genotypes can be accompanied by adverse effects, and contraindications to therapy are not infrequent (McHutchison and Patel (2002) Hepatology, 36: S245-252). Standard antiviral combination therapy with interferon has been shown to be an effective secondary prevention of HCC (El-Serag (2004) Gastroenterology, 127: S27-34); however, 70-80% of HCV patients are not candidates for these therapies (Butt et al. (2007) Hepatology, 46: 364A; Falck-Ytter et al. (2002) Annl. Intern. Med., 136: 288-292). N S3/4A protease inhibitors in combination with PEG-IFN and ribavirin have recently been approved and show increased SVR, but also increased adverse events including anemia and gastrointestinal symptoms (Ciesek et al. (2011) Gastroenterol. & Hepatol., 8: 69-71). Since these therapies still require interferon, a significant population of patients cannot receive this treatment. Current approaches for preventing HCV-related HCC in patients with contraindications to standard therapy are limited.

### SUMMARY

In certain embodiments this novel antiviral peptide(s) are provided that are effective against positive sense RNA viruses that have an internal ribosome entry site IRES). The peptide(s) can be used to inhibit propagation of such viruses and thereby provide an effective modality for the treatment of infections such as hepatitis C, and the like.

In certain embodiments the peptide(s) range in length from about 8 amino acids up to about 100 amino acids (o up to about 75 amino acids, or up to about 50 amino acids, or up to about 45 amino acids, or up to about 40 amino acids, or up to about 35 amino acids, or up to about 34 amino acids, or up to about 30 amino acids, or up to about 25 amino acids, or up to about 20 amino acids, or up to about 15 amino acids, or up to about 14 amino acids) and comprises a domain having the amino acid sequence of the 13-hairpin of the C34 region of NS5A or conservative substitutions thereof, or is a mimetic of this domain. In certain embodiments the peptide comprises an amino acid sequence according to the formula X⁴-X⁵-X⁶-X⁷-X⁸-X⁹-X¹⁰⁻X¹¹, or the retro, inverso, or retro-inverso form of this amino acid sequence where X⁴ is an amino acid selected from the group consisting of Phe, ²Nal, Dpa, and Trp, or conservative substitutions thereof, X⁵ is an amino acid selected from the group consisting of Leu, and Arg, or conservative substitutions thereof, X⁶ is an amino acid selected from the group consisting of Val, Chg, Cys, and Cys^{tBu} (or Cys), or conservative substitutions thereof, X⁷ is an amino acid selected from the group consisting of Gly, Pro, and (D)Pro (or Pro), or conservative substitutions thereof, X⁸ is an amino acid selected from the group consisting of Leu, and Cha, or conservative substitutions thereof, X⁹ is an amino acid selected from the group consisting of Asn, and H is, or conservative substitutions thereof, X¹⁰ is an amino acid selected from the group consisting of Gln, Glu, and Arg, or conservative substitutions thereof, and X¹¹ is an amino acid selected from the group consisting of Tyr, Bip, and Dpa, or conservative substitutions thereof.

In certain embodiments the peptide comprises an amino acid sequence according to the formula X¹ₘ-X²ₙ-X³-X⁴-X⁵-X⁶-X⁷-X⁸-X⁹-X¹⁰-X¹¹-X¹²-X¹³ₒ-X¹⁴ₚ or the retro, inverso, or retro-inverso form of this amino acid sequence where m, n, o, and p are independently 0 or 1; X¹ is present or absent and when present is Arg, or a conservative substitution thereof; X² is present or absent and when present is an amino acid sequence selected from the group consisting of Val, and Cys, or conservative substitutions thereof; X³ is an amino acid selected from the group consisting of Thr, Ser, and Arg, or conservative substitutions thereof; X⁴ is an amino acid selected from the group consisting of Phe, ²Nal, Dpa, and Trp, or conservative substitutions thereof; X⁵ is an amino acid selected from the group consisting of Leu, and Arg, or conservative substitutions thereof; X⁶ is an amino acid selected from the group consisting of Val, Chg, Cys, and Cys^{tBu}, or conservative ubstitutions thereof; X⁷ is an amino acid selected from the group consisting of Gly, Pro, and (D)Pro, or conservative substitutions thereof; X⁸ is an amino acid selected from the group consisting of Leu, and Cha, or conservative substitutions thereof; X⁹ is an amino acid selected from the group consisting of Asn, and His, or conservative substitutions thereof; X¹⁰ is an amino acid selected from the group consisting of Gln, Glu, and Arg, or conservative substitutions thereof; X¹¹ is an amino acid selected from the group consisting of Tyr, Bip, and Dpa, or conservative substitutions thereof; X¹² is an amino acid selected from the group consisting of Leu, Pro, and Arg, or conservative substitutions thereof; X¹³ is present or absent and when present is an amino acid selected from the group consisting of Val, and Cys, or conservative substitutions thereof; and X¹⁴ is present or absent and, when present is an Arg or a conservative substitution thereof. In certain embodiments X⁴ is Phe or a conservative substitution thereof; and/or X⁶ is Val or a conservative substitution thereof; and/or X⁸ is Leu or a conservative substitution thereof; and/or X¹⁰ is Tyr, or a conservative substitution thereof. In certain embodiments X⁴ is selected from the group consisting of Dpa, ²Nal and Trp; and/or X⁶ is selected from the group consisting of Chg, and Cys(tBut); and/or X¹¹ is selected from the group consisting of Bip and Dpa. In certain embodiments the amino acid sequence of said peptide comprises a sequence selected from the group consisting of Phe-Leu-Val-Gly-Leu-Asn-Gln-Tyr (SEQ ID NO:6), Phe-Arg-Val-Gly-Leu-His-Glu-Tyr (SEQ ID NO:7), Phe-Arg-Val-Gly-Leu-His-Glu-Tyr (SEQ ID NO:8), Phe-Arg-Val-Pro-Cha-His-Glu-Tyr (SEQ ID NO:9), Phe-Arg-Val-Pro-Cha-His-Arg-Tyr (SEQ ID NO:10), Phe-Arg-Val-Pro-Cha-His-Arg-Tyr (SEQ ID NO:11), Phe-Arg-Val-Pro-Cha-His-Arg-Bip (SEQ ID NO:12), Phe-Arg-Val-Pro-Cha-His-Arg-Dpa (SEQ ID NO:13), Phe-Arg-Chg-Pro-Cha-His-Arg-Tyr (SEQ ID NO:14), Phe-Arg-Cys^{StBu}-Pro-Cha-His-Arg-Tyr (SEQ ID NO:15), ²Nal-Arg-Val-Pro-Cha-His-Arg-Tyr (SEQ ID NO:16), Dpa-Arg-Val-Pro-Cha-His-Arg-Tyr (SEQ ID NO:17), Trp-Arg-Val-Pro-Cha-His-Arg-Dpa (SEQ ID NO:18), ²Nal-Arg-Chg-Pro-Cha-His-Arg-Bip (SEQ ID NO:19), ²Nal-Arg-Val-Pro-Cha-His-Arg-Tyr (SEQ ID NO:20), ²Nal-Arg-Val-Pro-Cha-His-Arg-Tyr (SEQ ID NO:21), ²Nal-Arg-Val-Pro-Cha-His-Arg-Tyr (SEQ ID NO:22), ²Nal-Arg-Val-Pro-Cha-His-Arg-Tyr (SEQ ID NO:23), ²Nal-Arg-Chg-Pro-Cha-His-Arg-Tyr (SEQ ID NO:24), ²Nal-Arg-Chg-Pro-Cha-His-Arg-Tyr (SEQ ID NO:25)), or conservative substitutions of one, two, or three residues comprising said sequences. In certain embodiments the amino acid sequence of the peptide comprises (or consists of) a sequence selected from the group consisting of Val-Thr-Phe-Leu-Val-Gly-Leu-Asn-Gln-Tyr-Leu-Val (SEQ ID NO:26), Val-Ser-Phe-Arg-Val-Gly-Leu-His-Glu-Tyr-Pro-Val (SEQ ID NO:27), Tyr-Phe-Val-Pro-His-Glu-Ser-Gly-Arg-Val-Val-Leu (SEQ ID NO:28), Cys-Ser-Phe-Arg-Val-Gly-Leu-His-Glu-Tyr-Pro-Cys (SEQ ID NO:29), Cys-Ser-Phe-Arg-Val-Pro-Cha-His-Glu-Tyr-Pro-Cys (SEQ ID NO:30), Arg-Cys-Arg-Phe-Arg-Val-Pro-Cha-His-Arg-Tyr-Arg-Cys-Arg (SEQ ID NO:31), Arg-Cys-Arg-Phe-Arg-Val-Pro-Cha-His-Arg-Tyr-Arg-Cys-Arg (SEQ ID NO:32), Arg-Cys-Arg-Phe-Arg-Val-Pro-Cha-His-Arg-Bip-Arg-Cys-Arg (SEQ ID NO:33), Arg-Cys-Arg-Phe-Arg-Val-Pro-Cha-His-Arg-Dpa-Arg-Cys-Arg (SEQ ID NO:34), Arg-Cys-Arg-Phe-Arg-Chg-Pro-Cha-His-Arg-Tyr-Arg-Cys-Arg (SEQ ID NO:35), Arg-Cys-Arg-Phe-Arg-Cys-Pro-Cha-His-Arg-Tyr-Arg-Cys-Arg (SEQ ID NO:36), Arg-Cys-Arg-²Nal-Arg-Val-Pro-Cha-His-Arg-Tyr-Arg-Cys-Arg (SEQ ID NO:37), Arg-Cys-Arg-Dpa-Arg-Val-Pro-Cha-His-Arg-Tyr-Arg-Cys-Arg (SEQ ID NO:38), Arg-Cys-Arg-Trp-Arg-Val-Pro-Cha-His-Arg-Dpa-Arg-Cys-Arg (SEQ ID NO:39), Arg-Cys-Arg-²Nal-Arg-Chg-Pro-Cha-His-Arg-Bip-Arg-Cys-Arg (SEQ ID NO:40), Cys-Arg-²Nal-Arg-Val-Pro-Cha-His-Arg-Tyr-Arg-Cys (SEQ ID NO:41), Cys-Arg-²Nal Nal-Arg-Val-Pro-Cha-His-Arg-Tyr-Arg-Cys (SEQ ID NO:42), Arg-²Nal-Arg-Val-Pro-Cha-His-Arg-Tyr-Arg-Cys (SEQ ID NO:43), Arg-Nal-Arg-Val-Pro-Cha-His-Arg-Tyr-Arg (SEQ ID NO:44), Cys-Arg-²Nal-Arg-Chg-Pro-Cha-His-Arg-Tyr-Arg-Cys (SEQ ID NO:45), and Arg-Nal-Arg-Chg-Pro-Cha-His-Arg-Tyr-Arg (SEQ ID NO:46), or conservative substitutions of one, two, or three residues comprising said sequences. In various embodiments any of these peptides comprise one or more “D” amino acids. In certain embodiments any of the prolines is a (D) proline. In certain embodiments wherein X⁷ comprises a (D)proline. In certain embodiments the peptide comprises all “D” amino acids. In certain embodiments the acid sequence of said peptide comprises the amino acid sequence of residues X⁴-X¹¹ as shown in Table 1 or FIG. 9. In certain embodiments the acid sequence of said peptide comprises the amino acid sequence of residues X²-X¹² as shown in Table 1 or FIG. 9. In certain embodiments the amino acid sequence of the peptide comprises an amino acid sequence of a peptide shown in Table 1 or FIG. 9 (e.g., the amino acid sequence of HCV1, HCV3, HCV4, HCV5, HCV6, HCV7, HCV8, HCV9, HCV10, HCV11, HCV12, HCV13, HCV14, HCV15, HCV15R, HCV16, HCV17, HCV18, HCV19, and/or HCV18RI). In certain embodiments the amino acid sequence of the peptide consists of an amino acid sequence of a peptide shown in Table 1 or FIG. 9. In certain embodiments the peptide is a peptide shown in Table 1 or FIG. 9. In certain embodiments the peptide comprises all “L” amino acids. In certain embodiments the peptide comprises the inverso of any of the foregoing amino acid sequences. In certain embodiments the peptide comprises the retro form of any of the foregoing amino acid sequences. In certain embodiments the peptide comprises the retro-inverso form of any of the foregoing amino acid sequences. In certain embodiments the peptide further comprises a cell penetrating peptide attached to the amino or carboxyl terminus. In certain embodiments the cell penetrating peptide comprises a poly Arg tag of the form R-(Ahx-R)₆. In certain embodiments the cell penetrating peptide comprises the amino acid sequence of a peptide shown in Table 2. In certain embodiments the peptide is a beta peptide. In certain embodiments the peptide forms a beta hairpin conformation. In certain embodiments the peptide is a cyclized peptide. In certain embodiments the peptide can be “cyclized” with a disulfide bridge, thioether linkage, or other linker, e.g., to stabilize the 13-hairpin conformation. When present the “cyclizing” linker can connect the two terminal amino acids of the peptide, two internal amino acids of the peptide, or an internal amino acid and a terminal amino acid of the peptide. In certain embodiments the peptide comprises a disulfide (—S—S—) bond or a thioether (—S—CH₂—) bridges. In certain embodiments the peptide lacks an inter-amino acid linkage other than a peptide bond. In certain embodiments the peptide bears a first protecting group on the carboxyl terminus and/or a second protecting group on the amino terminus. In certain embodiments the first protecting group when present, and/or said second protecting group when present is protecting group selected from the group consisting of acetyl, amide, and 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (Bz10), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), t-butoxycarbonyl (Boc), cyclohexyloxy (cHxO),t-butoxymethyl (Bum), t-butoxy (tBuO), t-Butyl (tBu), Acetyl (Ac), and Trifluoroacetyl (TFA). In certain embodiments the first protecting group is present and is an amide. In certain embodiments the second protecting group is present and is an acetyl. In certain embodiments the peptide is pegylated. In certain embodiments the peptide is formulated as a pharmaceutical formulation. In certain embodiments the peptide is formulated in a lipid or liposome. In certain embodiments the peptide is formulated with a non-covalent carrier. In certain embodiments the peptide is formulated with a non-covalent carrier selected from the group consisting of pep-1 (Chariot), R(8), and azo-R(8). In certain embodiments the peptide is formulated with a recombinant vault nanocapsule. In certain embodiments the peptide is formulated as a unit dosage formulation. In various embodiments the peptide is not a full-length NS5A. In certain embodiments the peptide is not a full length C34 region of NS5A.

In certain embodiments pharmaceutical formulations are provided. The formulations typically comprises one or more peptides described herein and a pharmaceutically acceptable excipient. In certain embodiments the formulation is formulated for administration via a route selected from the group consisting of isophoretic delivery, transdermal delivery, aerosol administration, administration via inhalation, oral administration, intravenous administration, vaginal administration, and rectal administration. In certain embodiments the formulation is a unit dosage formulation.

In certain embodiments the peptides (and/or formulations) described herein can be used to inhibit propagation of positive sense RNA viruses that have an internal ribosome entry site (IRES) (e.g., Hepatitis C, Hepatitis A, various Flaviviridae, etc.) and/or as a prophylactic or therapeutic treatment for subjects that have been infected with such viruses or that are believed to be infected or at risk for infection with such viruses.

In certain embodiments a method of inhibiting intracellular production of a positive sense RNA virus that has an internal ribosome entry site (IRES) is provided. The method typically involves delivering to a cell containing the virus one or more antiviral peptides as described herein in an amount sufficient to reduce or block production of said virus.

In certain embodiments a method of inhibiting NS5A-mediated IRES activity and viral infection is provided. The method typically involves delivering to a cell containing the virus one or more antiviral peptides as described herein in an amount sufficient to reduce of block NS5A-mediated IRES activity of the virus. In various embodiments the delivering comprises administering the peptide(s) or causing the peptide(s) to be administered to a mammal infected with the virus.

In certain embodiments a method of treating a subject infected with a positive sense RNA virus that has an internal ribosome entry site (IRES) is provided. The method typically involves administering, or causing to be administered, to the subject one or more antiviral peptides as described herein in an in an amount to reduce or block propagation of the virus. In certain embodiments a method of prophylactically treating a subject believed to be infected or at significant risk of infection with a positive sense RNA virus that has an internal ribosome entry site (IRES) is provided. The method typically involves administering, or causing to be administered, to the subject one or more antiviral peptides as described herein in an in an amount to reduce or block propagation of the virus. In various embodiments the delivering or administering comprises administering said peptide via a route selected from the group consisting of oral administration, nasal administration, rectal administration, intraperitoneal injection, intravascular injection, subcutaneous injection, transcutaneous administration, inhalation administration, and intramuscular injection. In certain embodiments the virus is a member of the Picornaviridae (e.g., Hepatitis A virus, Hepatitis C virus, Rhinovirus, Poliovirus, Echovirus, Coxsackievirus A, and B, Mengovirus, etc.). In certain embodiments the virus is Hepatitis C. In certain embodiments the virus is a member of the Flaviviridae (e.g., Yellow fever virus, West Nile virus, Dengue fever virus, etc.). In certain embodiments the virus is a member of the Caliciviridae (e.g., Norwalk virus).

In certain embodiments a peptide (or pharmaceutical formulation) as described herein is provided for use in: inhibiting intracellular production of a positive sense RNA virus that has an internal ribosome entry site (IRES); and/or inhibiting NS5A-mediated IRES activity and viral infection; and/or treating a subject infected with a positive sense RNA virus that has an internal ribosome entry site (IRES). In certain embodiments the virus is a member of the Picornaviridae. In certain embodiments virus is hepatitis A virus or a Hepatitis C virus. In certain embodiments the virus is Hepatitis C virus. In certain embodiments the virus is selected from the group consisting of Rhinovirus, Poliovirus, Hepatitis A virus, Echovirus, Coxsackievirus A, and B, Mengovirus. In certain embodiments the virus is a member of the Flaviviridae. In certain embodiments the virus is selected from the group consisting of Yellow fever virus, West Nile virus, and Dengue fever virus. In certain embodiments the virus is a member of the Caliciviridae (e.g., Norwalk virus).

In certain embodiments C34 region of NS5A or preferably the β-hairpin of the C34 region of NS5A can be used as targets to screen for moieties that peptides can be used to inhibit propagation of positive sense RNA viruses that have an internal ribosome entry site (IRES) and/or for the treatment or prophylaxis of infections by such viruses. In certain embodiments the screening methods involve contacting the C34 peptide or a peptide comprising 13-hairpin region of the C34 peptide with one or more test agents. Agents that bind or interact with the C34 peptide and more preferably with the 13-hairpin are good candidate agents that can be used to inhibit propagation of positive sense RNA viruses that have an internal ribosome entry site (IRES) and/or for the treatment or prophylaxis of infections by such viruses.

### DEFINITIONS

The following are certain abbreviations used herein: HCV, hepatitis C virus; HCC, hepatocellular carcinoma; PEG-IFN, pegylated interferon-α; SVR, sustained virologic response; NS5A, non-structural protein 5A; NCR, non-coding region; IRES, internal ribosomal entry site; HSP, heat shock protein; PCR, polymerase chain reaction; His6, hexa-histidine; ASM, alanine scan mutagenesis; GST, glutathione S-transferase; Fmoc, 9-fluorenylmethyloxycarbonyl; RP-HPLC, reverse-phase high performance liquid chromatography; MALDI-MS, matrix-assisted laser desorption ionization spectrometry; ORF, open reading frame; C34, C-terminal 34 amino acids of NS5A domain I; HCVcc, HCV cell culture; NBD, nucleotide binding domain; SBD, substrate binding domain; FLuc, Firefly luciferase; RLuc, Renilla luciferase.

An internal ribosome entry site, abbreviated IRES, is a nucleotide sequence that allows for translation initiation in the middle of a messenger RNA (mRNA) sequence as part of the greater process of protein synthesis. Internal ribosome entry sites are known for a wide variety of organisms (see, e.g., IRESite: the database of experimentally verified IRES structures (www.iresite.org), and the like).

The term “peptide” as used herein refers to a polymer of amino acid residues typically ranging in length from 2 to about 50 or about 60 residues. In certain embodiments the peptide ranges in length from about 6, 7, 8, or, 9 residues up to about 60, 50, 45, 40, 45, 30, 25, 20, 15, or 14 residues. In certain embodiments the amino acid residues comprising the peptide are all “L-form” amino acid residues, however, it is recognized that in various embodiments, “D” amino acids can be incorporated into the peptide. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an α-ester, a β-ester, a thioamide, phosphonamide, carbomate, hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “residue” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, n-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, (L) Cyclohexylalanine, (L)-tert-Leucine, L)-Cyclohexylglycine, Pentafluoro-(L)-phenylalanine, 3,3-Diphenyl-(L)-alanine, (L)-Biphenylalanine, 3-(1-Naphthyl)-(L)-alanine, 3-(2-Naphthyl)-(L)-alanine, 6-aminohexanoic acid, palmitic acid, and the like. These modified amino acids are illustrative and not intended to be limiting.

An L-peptide has three analogue sequences (enantiomers) built from L and D amino acids: the D-enantiomer or inverso-peptide with the same sequence, but composed of D-amino acids and a mirror conformation (it will be recognized that the “L” peptide is the inverso of the corresponding “D” peptide); the retro-peptide, consisting of the same sequence of amino acids but in reverse order; and the retro-inverso or D-retro-enantiomer peptide, consisting of D-amino acids in the reversed sequence.

“β-peptides” comprise “βamino acids”, which have their amino group bonded to the β carbon rather than the α-carbon as in the 20 standard biological amino acids.

Peptoids, or N-substituted glycines, are a specific subclass of peptidomimetics. They are closely related to their natural peptide counterparts, but differ chemically in that their side chains are appended to nitrogen atoms along the molecule's backbone, rather than to the α-carbons (as they are in natural amino acids).

In certain embodiments, conservative substitutions of the amino acids comprising any of the sequences described herein are contemplated. In various embodiments one, two, three, four, or five different residues are substituted. The term “conservative substitution” is used to reflect amino acid substitutions that do not substantially reduce the activity (e.g., antiviral activity) of the molecule. Typically conservative amino acid substitutions involve substitution one amino acid for another amino acid with similar chemical properties (e.g. charge or hydrophobicity). Certain conservative substitutions include “analog substitutions” where a standard amino acid is replaced by a non-standard (e.g., rare, synthetic, etc) amino acid differing minimally from the parental residue. Amino acid analogs are considered to be derived synthetically from the standard amino acids without sufficient change to the structure of the parent, are isomers, or are metabolite precursors. Examples of such “analog substitutions” include, but are not limited to, 1) Lys-Orn, 2) Leu-Norleucine, 3) Lys-Lys[TFA], 4) Phe-Phe[Gly], and 5) 6-amino butylglycine-ξ-amino hexylglycine, where Phe[gly] refers to phenylglycine(a Phe derivative with a H rather than CH₃ component in the R group), and Lys[TFA] refers to a Lys where a negatively charged ion (e.g., TFA) is attached to the amine R group. Other conservative substitutions include “functional substitutions” where the general chemistries of the two residues are similar, and can be sufficient to mimic or partially recover the function of the native peptide. Strong functional substitutions include, but are not limited to 1) Gly/Ala, 2) Arg/Lys, 3) Ser/Tyr/Thr, 4) Leu/Ile/Val, 5) Asp/Glu, 6) Gln/Asn, and 7) Phe/Trp/Tyr, while other functional substitutions include, but are not limited to 8) Gly/Ala/Pro, 9) Tyr/His, 10) Arg/Lys/His, 11) Ser/Thr/Cys, 12) Leu/Ile/Val/Met, and 13) Met/Lys (special case under hydrophobic conditions). Various “broad conservative substations” include substitutions where amino acids replace other amino acids from the same biochemical or biophysical grouping. This is similarity at a basic level and stems from efforts to classify the original 20 natural amino acids. Such substitutions include 1) nonpolar side chains: Gly/Ala/Val/Leu/Ile/Met/Pro/Phe/Trp, and/or 2) uncharged polar side chains Ser/Thr/Asn/Gln/Tyr/Cys. In certain embodiments broad-level substitutions can also occur as paired substitutions. For example, Any hydrophilic neutral pair [Ser, Thr, Gln, Asn, Tyr, Cys]+[Ser, Thr, Gln, Asn, Tyr, Cys] can may be replaced by a charge-neutral charged pair [Arg, Lys, His]+[Asp, Glu]. The following six groups each contain amino acids that, in certain embodiments, are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K), Histidine (H); 5) Isoleucine (I), Leucine (L), Methionine (M), Valine (V); and 6) Phenylalanine (F), Tyrosine (Y), Tryptophan (W). Where amino acid sequences are disclosed herein, amino acid sequences comprising, one or more of the above-identified conservative substitutions are also contemplated.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

As used herein, “administering” refers to local and systemic administration, e.g., including enteral, parenteral, pulmonary, and topical/transdermal administration. Routes of administration for the antiviral peptide(s) described herein that find use in the methods described herein include, but are not limited to, oral (per os (p.o.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The term “effective amount” or “pharmaceutically effective amount” refer to the amount and/or dosage, and/or dosage regime of one or more compounds necessary to bring about the desired result e.g., an amount sufficient to reduce or block propagation of a virus (e.g., hepatitis C), or an amount sufficient to lessen the severity or delay the progression of a symptoms of the viral disease (e.g., therapeutically effective amounts), an amount sufficient to reduce the risk or delaying the onset, and/or reduce the ultimate severity of a disease caused by a viral infection (e.g., prophylactically effective amounts).

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

The terms “subject”, “individual”, and “patient” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments, the subject may not be under the care or prescription of a physician or other health worker.

The terms “treatment”, “treating”, or “treat” as used herein, refer to actions that produce a desirable effect on the symptoms or pathology of a disease or condition, particularly those that can be effected utilizing the peptides described herein, and may include, but are not limited to, even minimal changes or improvements in one or more measurable markers of the disease or condition being treated. Treatments also refers to delaying the onset of, retarding or reversing the progress of, reducing the severity of, or alleviating or preventing either the disease or condition to which the term applies, or one or more symptoms of such disease or condition. “Treatment”, “treating”, or “treat” does not necessarily indicate complete eradication or cure of the disease or condition, or associated symptoms thereof. In one embodiment, treatment comprises improvement of at least one symptom of a disease being treated. The improvement may be partial or complete. The subject receiving this treatment is any subject in need thereof. Exemplary markers of clinical improvement will be apparent to persons skilled in the art.

### BRIEF DESCRIPTION OF THE DRAWINGS

FIG. 1 shows relative antiviral activity of the majority of the antiviral peptides shown in Table 1. Cells were infected with reporter hepatitis c virus then treated with indicated peptide at a concentration of 10 nm. Cells were harvested 24 hours post infection and viral production was measured by luciferase assay.

FIG. 2, panels A-C, show that NS5A domain I directly binds HSP70 nucleotide binding domain (NBD). The upper part of all panels displays Western analysis of GST pull-down targets using antibody against NS5A (2A), HSP70 (2B), and the V5 epitope (2C). The lower part of all panels represents Coomassie stains showing GST-fusion bait proteins.

FIG. 3, panels A-F, show that the C-terminal region of NS5A domain I is necessary and sufficient for its interaction with HSP70. The upper part of all panels displays Western analysis of GST pull-down targets using antibody against the V5 epitope. The lower part of all panels represents Coomassie stains showing GST-fusion bait proteins. Target proteins are listed above each panel. Arrows indicate presence of target polypeptides. Panel A: Deletion of NS5A domain I abolishes its HSP70 interaction. Panel B: Deletion of NS5A membrane anchoring domain does not affect HSP70 binding. Panel C: Only NS5A domain I binds HSP70. Panel D: The N-terminal region of NS5A domain I is not necessary for HSP70 binding. Panel E: The C-terminal 34 amino acids of NS5A domain I (C34) are necessary for HSP70 interaction. Panel F: The C-terminal 34 amino acids of NS5A domain I (C34) are sufficient for HSP70 binding.

FIG. 4, panels A-C, show schematics of HCV genome (panel A), NS5A (panel B), and all NS5A subclones used in GST pull-down analyses in FIG. 3 (panel C). (+) and (−) indicate interaction and lack of interaction with HSP70, respectively.

FIGS. 5A-5D show that the hairpin structure at C-terminus of NS5A domain I regulates HCV IRES activity. FIG. 5A: Schematic of the bicistronic reporter construct used to measure IRESmediated translation. Firefly luciferase (FLuc) and Renilla luciferase (RLuc) are driven by HCV IRES and a 5′ cap, respectively. Each reporter has a stop codon to allow independent expression. Firefly to Renilla ratios are used to follow changes in levels of IRES-mediated translation. For all experiments, the IRES reporter, either NS5A or GFP, and a peptide construct (if indicated) were transfected into cells.

FIG. 5B: A 34-amino acid peptide from C terminus of NS5A domain I (C34) suppresses the NS5A-driven increase in IRES-mediated translation. FIG. 5C: Triple-alanine scan mutagenesis (ASM) across the C34 region indicates that amino acids 171-179 of NS5A domain I are primarily involved in augmenting IRES-mediated translation. FIG. 5D: The HCV4 synthetic peptide derivative of C34 hairpin blocks NS5A-augmented IRES-mediated translation.

FIGS. 6A-6C show that C34 structure and hydrophobicity support its sequence-specific HSP70 interaction. FIG. 6A: The crystal structure of dimeric NS5A domain I as reported previously (Tellinghuisen et al. (2005) Nature, 435: 374-379). The two domain I monomers are shown in white, while the two C34 regions are in black. There are two exposed anti-parallel beta-sheets (hairpins) in each C34 region. FIG. 6B: A close-up view of one of the C34 regions where the most significant mutagenesis residues (FIG. 4, panel C) are colored in black suggesting that the C34 hairpin is the main site of NS5A/HSP70 interaction. FIG. 6C: Hydrophobicity plot of NS5A generated using the Kyte and Doolittle scale with a window size of seven. The location of C34 hairpin is indicated.

FIGS. 7A-7C show that the C34 hairpin regulates viral protein production. FIG. 7A: Renilla luciferase assay demonstrates that the C34 peptide from NS5A domain I significantly inhibits intracellular viral protein production. Huh-7.5 cells were transfected with the indicated peptide construct and 48 hours later infected with the reporter virus for three hours. Cells were harvested 24 hours post infection, and Renilla activity was measured. All Renilla values were normalized to GFP transfection. FIG. 7B: The C34 hairpin peptide derivative (HCV4) significantly suppresses intracellular viral protein translation. Huh-7.5 cells were treated with the indicated concentrations of HCV4 or the arginine tag control peptide and immediately infected with the Renilla reporter virus for three hours. Cells were assayed 24 hours post infection for Renilla activity. ‘Percent protection’ refers to the percentage of decrease in Renilla levels compared with no peptide treatment. FIG. 7C: MTT viability assay showing the cytotoxicity of the HCV4 peptide (arginine-tagged).

FIGS. 8A and 8B illustrate a proposed model of NS5A-augmented IRES-mediated translation. FIG. 8A: The data presented herein support the hypothesis that NS5A/HSP70 complex formation is important for NS5A-driven IRES-mediated translation. FIG. 8B: Soluble C34 hairpin peptide derivative inhibits NS5A/HSP70 binding resulting in inhibition of NS5A-driven IRES-mediated translation. 193×179 mm (300×300 DPI).

FIG. 9 illustrates various peptides that can be used to inhibit HCV infection (in various embodiments, excluding HCV2 which is a scrambled sequence). Crystal structure (SEQ ID NO:47), HCV1 (SEQ ID NO:48), HCV2 (SEQ ID NO:49), HCV3 (SEQ ID NO:50), HCV4 (SEQ ID NO:51), HCV5 (SEQ ID NO:52), HCV6 (SEQ ID NO:53), HCV7 (SEQ ID NO:54), HCV8 (SEQ ID NO:55), HCV9 (SEQ ID NO:56), HCV10 (SEQ ID NO:57), HCV11 (SEQ ID NO:58), HCV12 (SEQ ID NO:59), HCV13 (SEQ ID NO:60), HCV14 (SEQ ID NO:61), HCV15 (SEQ ID NO:62), HCV15R (SEQ ID NO:63), HCV16 (SEQ ID NO:64), HCV17 (SEQ ID NO:65), HCV18 (SEQ ID NO:66), HCV19 (SEQ ID NO:67), HCVR1 (SEQ ID NO:68).

### DETAILED DESCRIPTION

Hepatitis C virus (HCV) possesses a 10 kb positive sense RNA genome that encodes 10 viral proteins (Lindenbach and Rice (2005) Nature, 436: 933-938). The non-structural protein 5A (NS5A) is a 56-59 kDa phosphoprotein that associates with the viral replicase complex. It has been implicated in the regulation of HCV genome replication, viral protein translation, and virion assembly (Tellinghuisen et al. (2008) J. Virol., 82: 1073-1083; He et al. (2003) J. General Virol., 84: 535-543; Hughes et al. (2009) J. General Virol., 90: 1329-1334). NS5A also appears to modulate hepatocyte cell signaling by 1) promoting cell survival (Peng et al. (2010) J. Biol. Chem., 285: 20870-20881), 2) facilitating the viral life cycle (Tellinghuisen et al. (2008) J. Virol., 82: 1073-1083; He et al. (2003) J. General Virol., 84: 535-543; Hughes et al.,(2009) J. General Virol., 90: 1329-1334), and 3) interfering with the hepatocyte innate immune response (Kriegs et al. (2009) 284: 28343-28351).

The 5′ non-coding region (NCR) of HCV genome lacks a 5′ cap and instead possesses an internal ribosomal entry site (IRES) (Wang et al. (1993) J. Virol., 67: 3338-3344), a cis-acting element found in some host RNA transcripts as well as in viruses that allows ribosomal translation initiation to occur internally within a transcript in lieu of 5′ cap-dependent translation (Pacheco et al. (2010) J. Biomed. & Biotech. 2010: 458927). NS5A has been implicated in modulating HCV IRES-mediated translation (He et al. (2003) J. General Virol., 84: 535-543; Kalliampakou et al. (2005) J General Virol., 86: 1015-1025).

Through co-immunoprecipitation, a complex of NS5A and heat-shock proteins (HSPS) composed of NS5A, HSP70, and HSP40 (cofactor of HSP70) was identified and their colocalization in Huh-7 cells was demonstrated. It was further demonstrated that both NS5A-augmented IRES-mediated translation and virus production are blocked by HSP70 knockdown as well as by HSP synthesis inhibitor Quercetin, with no associated cytotoxicity.

It was hypothesized that HCV utilizes an NS5A/HSP complex to facilitate IRES-mediated translation of its genome. A NS5A/HSP70 interaction site is identified and it was demonstrated that a soluble peptide derivative of this site inhibits NS5A-mediated IRES activity and viral infection.

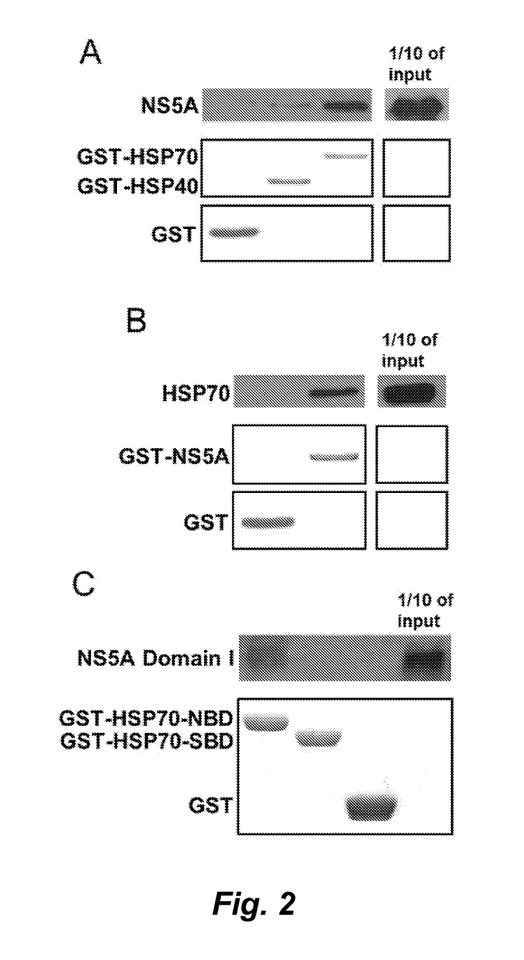

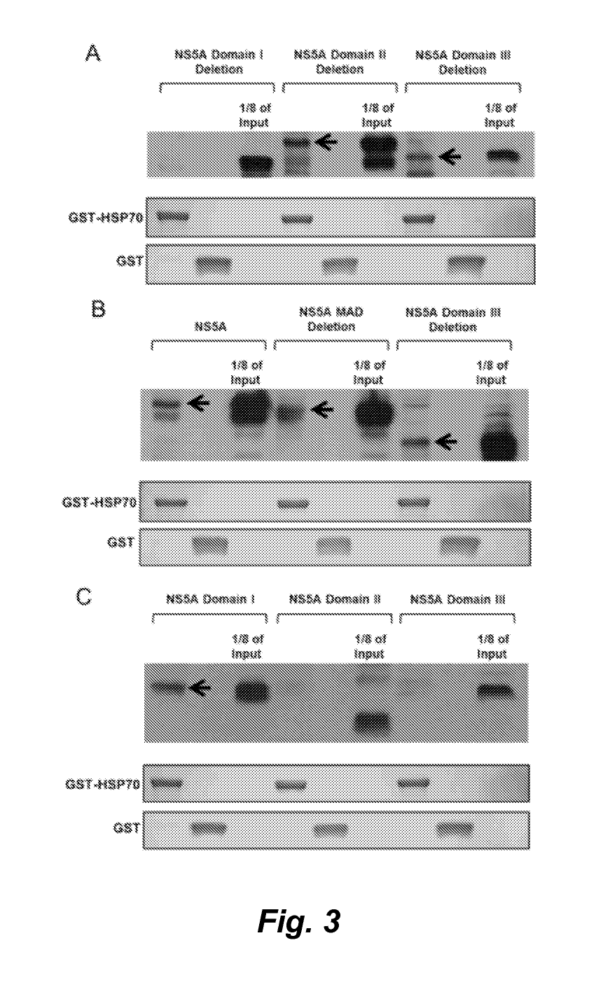

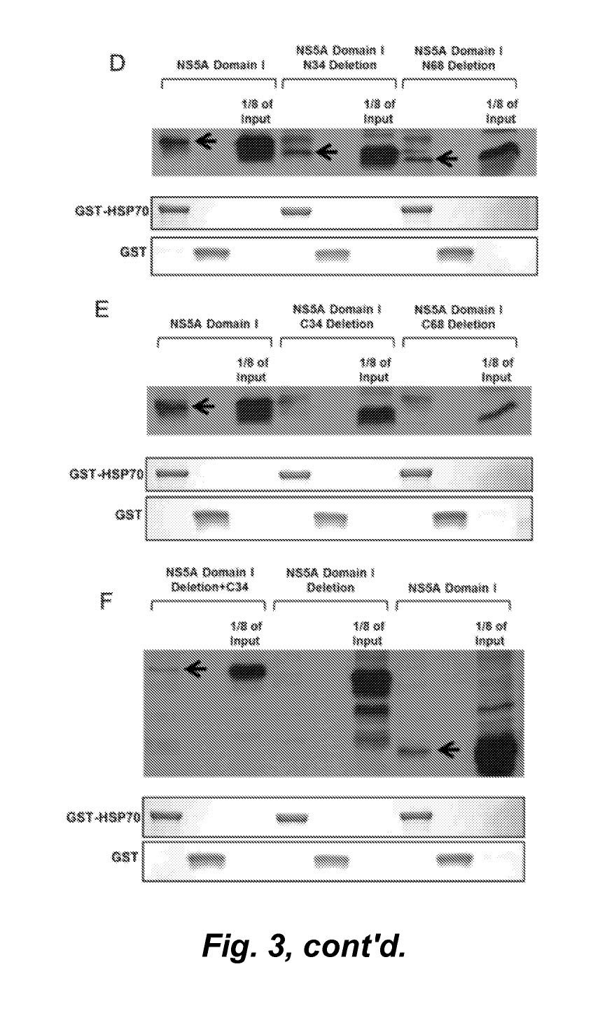

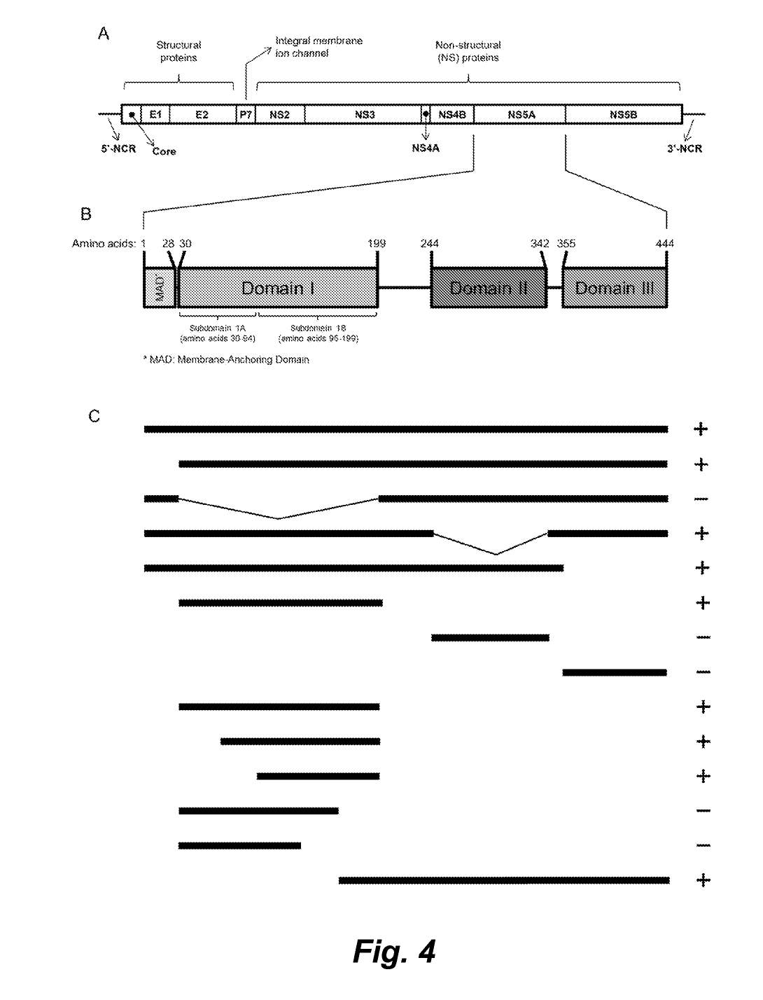

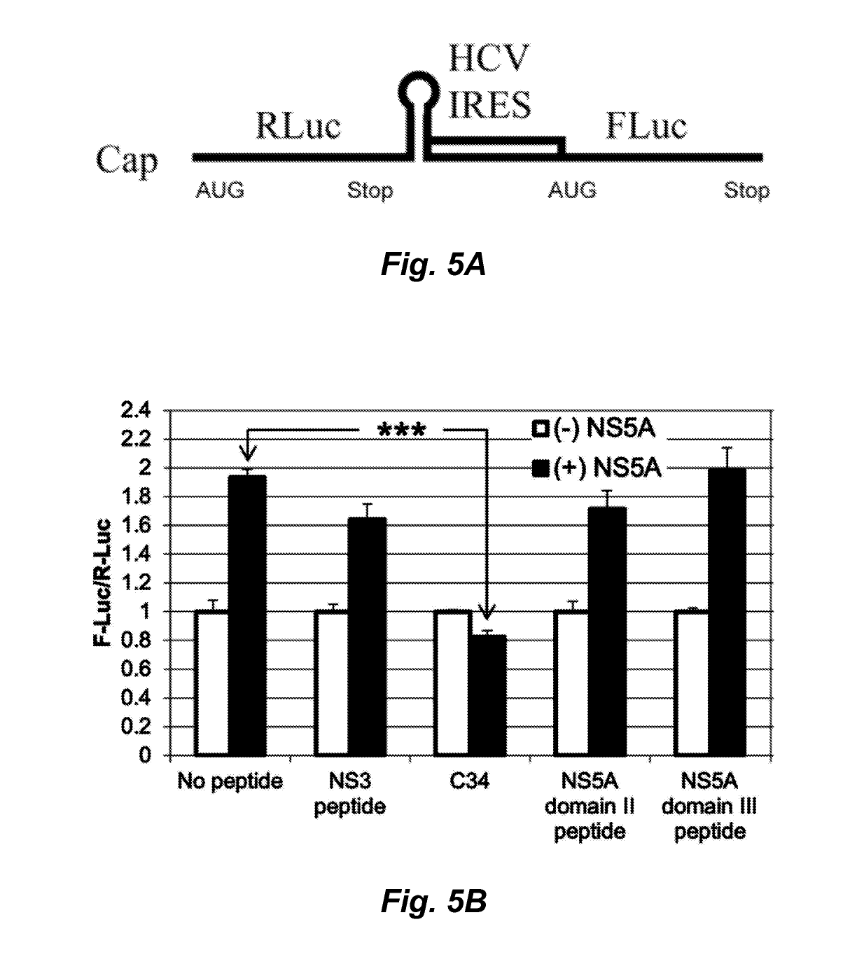

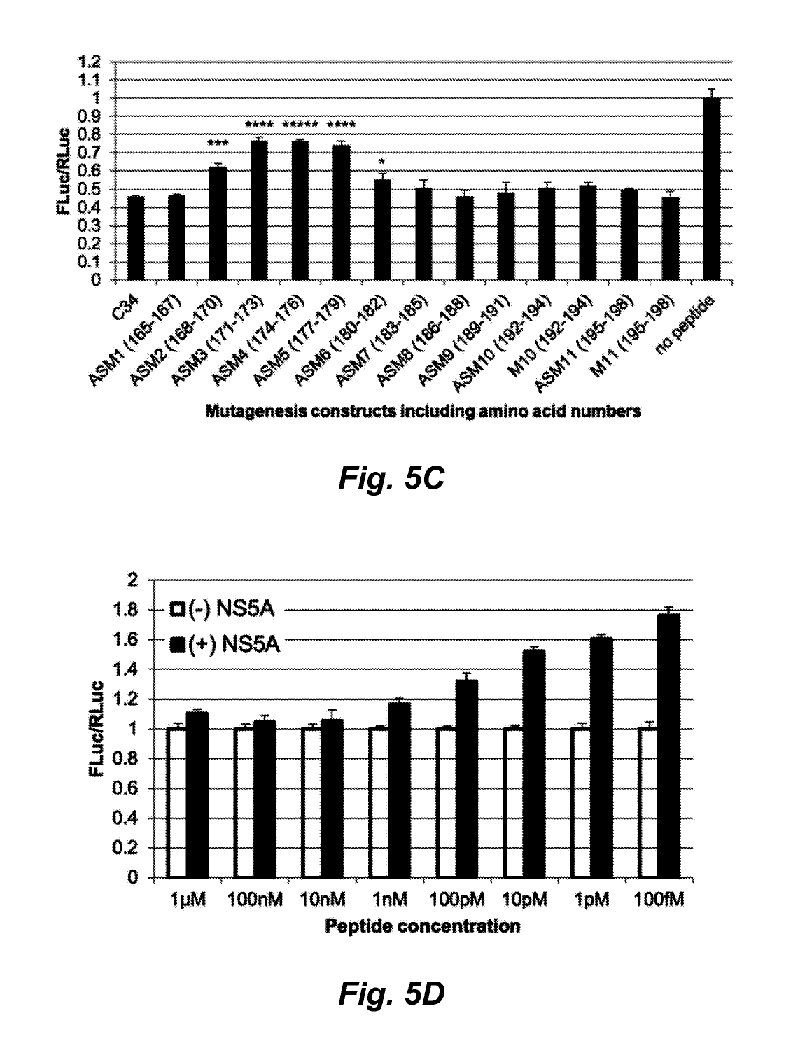

More particularly a 34 amino acid element (C34) in NS5A was identified and determined to be responsible for the interaction with HSP70 (see, e.g., FIG. 2). Introduction of C34 (LREEVSFRVGLHEYPVGSQLPCEP EPDVAVLTSM, SEQ ID NO:1, β-hairpin underlined) into the HCV cell culture (HCVcc) system reduced intracellular viral protein levels in contrast to control peptides of the same size from other areas of NS5A (see, e.g., FIG. 3). C34 also competitively inhibited NS5A-augmented IRES-mediated translation, while controls did not (FIG. 4). Through alanine scanning mutagenesis, the active motif within C34 was mapped to an exposed beta-sheet hairpin (see, e.g., underlined sequence above and FIGS. 5A-5C).

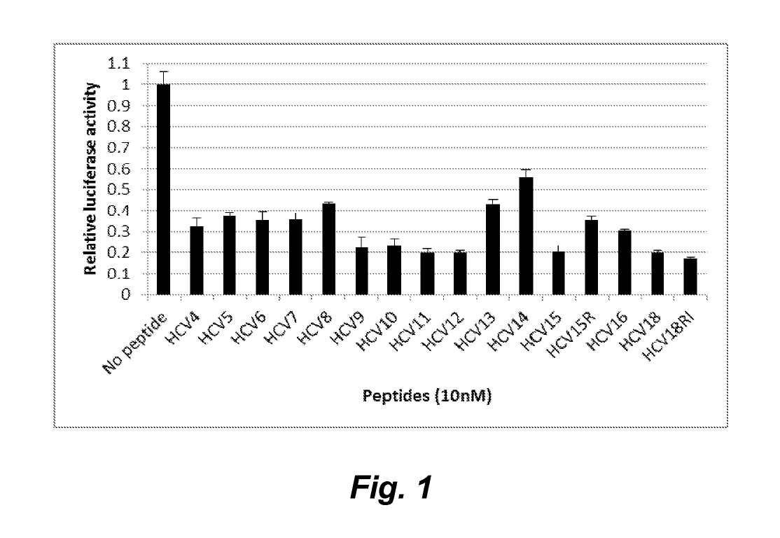

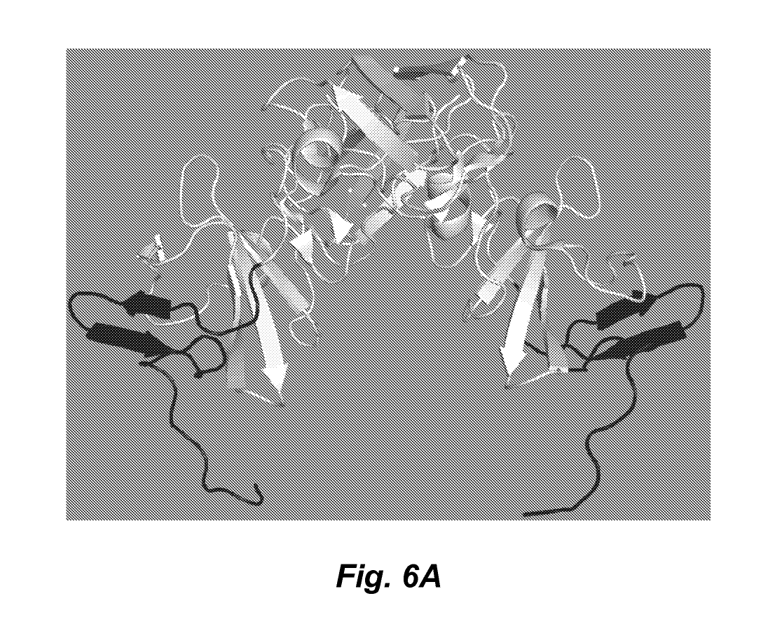

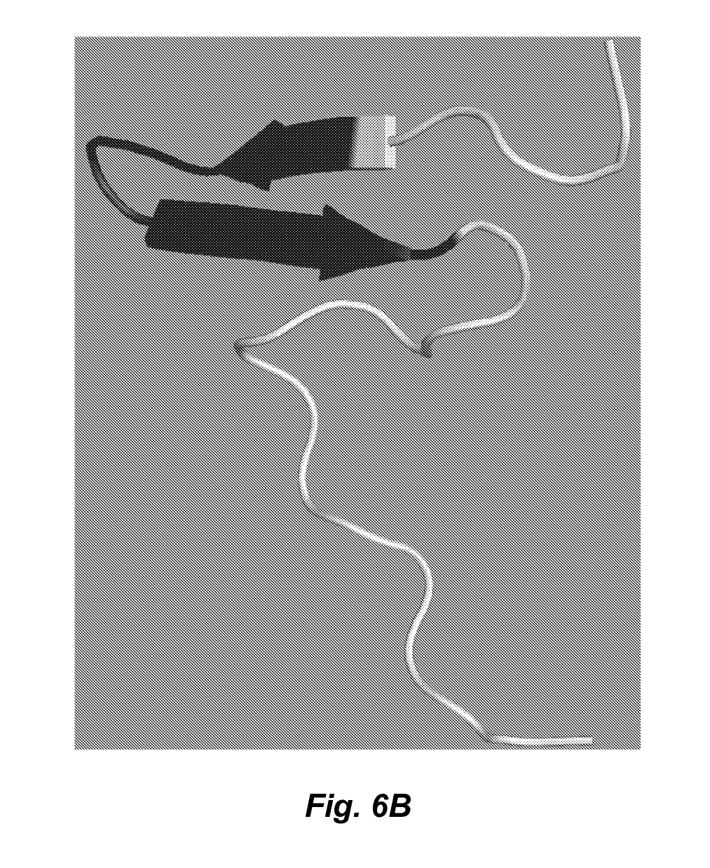

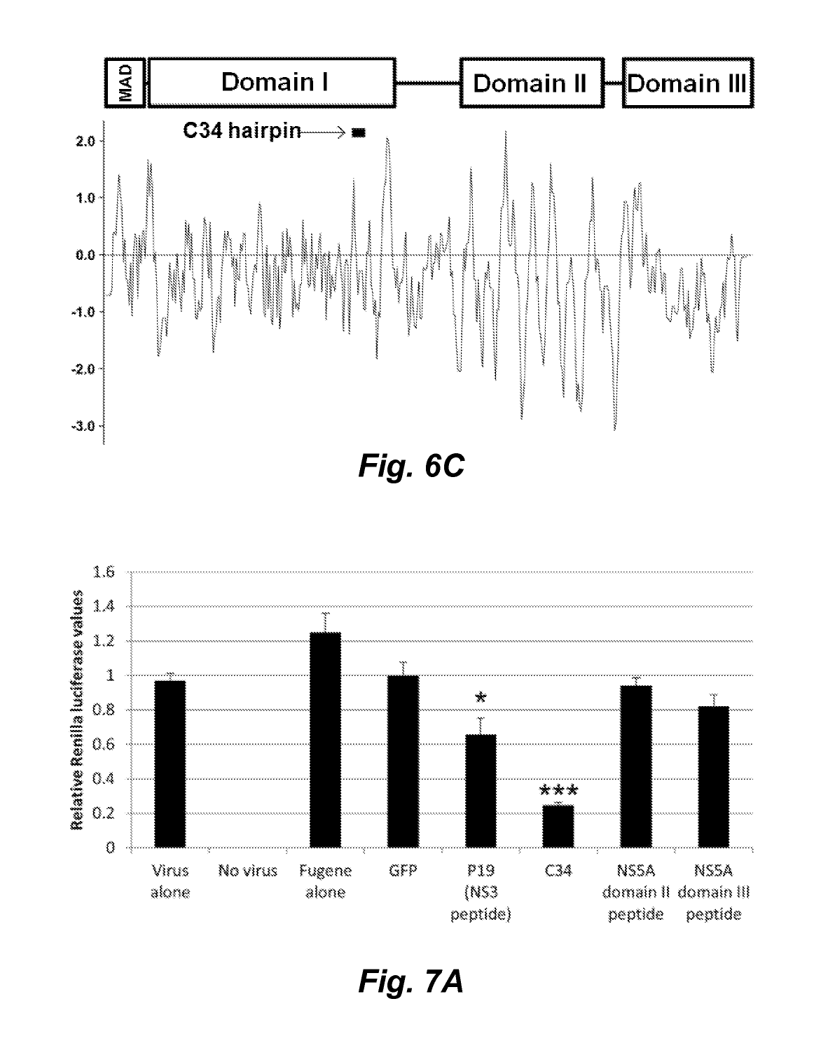

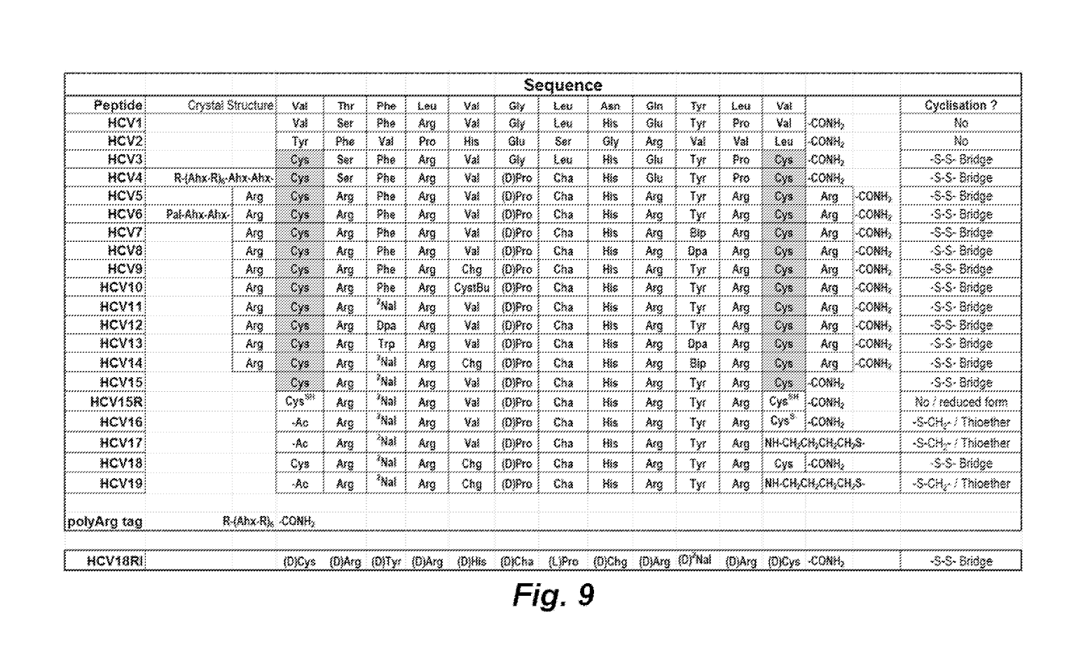

A 10 aa modified peptide corresponding to the C34 hairpin motif with an (optional) arginine tail (to facilitate cellular entry (HCV4, Table 1) was synthesized and this peptide was able to significantly inhibit intracellular virus production with no associated cellular toxicity (see, e.g., FIG. 6). Accordingly, it is believed that this peptide and derivatives (see, e.g., Table 1, FIG. 1, and FIG. 9) including, but not limited to retro forms of these peptides, inverso forms of these peptides, and retro-inverso forms of these peptides (as well as other peptides comprising a domain that mimics the C34 hairpin motif) can be used to therapeutically or prophylactically treat subjects with chronic hepatitis C and/or other viral infections as described herein. In certain embodiments subjects (e.g., humans or non-human mammals) with chronic hepatitis C infection are administered the peptide(s) (or derivatives), e.g., orally or intravenously, alone or in combination with other antiviral agents including, for example, current standard therapy to improve SVR. Alternatively the peptide(s) can be administered with antiviral agents in the absence of interferon thereby allowing subjects who can't receive or tolerate interferon to be treated.

Further, it is believed these peptides can also be used to treat other positive-sense RNA viruses that do or may possess an IRES including other Flaviviridae (Yellow fever virus, West Nile virus, Dengue fever virus), Picornaviridae (Rhinovirus, Poliovirus, Hepatitis A virus, Echovirus, Coxsackievirus A and B, Mengovirus), and Caliciviridae (Norwalk virus). Viruses (and other organisms) that possess an IRES are well known to those of skill in the art (see, e.g., Mokrej\check{s} et al. (2005) Nucleic Acids Res., 34(suppl 1): D125-D1301, and IRESite: the database of experimentally verified IRES structures (www.iresite.org)).

### Antiviral Peptides.

As indicated above, it was determined that the active motif β-hairpin of the C34 region of NS5A mediates the interaction with HSP70 and peptides comprising this region can be used to inhibit propagation of positive sense RNA viruses that have an internal ribosome entry site (IRES) (e.g., Hepatitis C, Hepatitis A, various Flaviviridae, etc.). Accordingly, in certain embodiments, peptides are contemplated that comprise a C34 β-hairpin domain (FRVGLHEYP, SEQ ID NO:2) or that comprise mimetics of that domain. In certain embodiments the peptides range in length from about 9 amino acids up to about 40 amino acids, in certain embodiments from about 9 or about 10 amino acids up to about 34 amino acids, more preferably from about 9 or about 10 amino acids up to about 30, or up to about 25, or up to about 20, or up to about 15, or up to about 14, or up to about 13, up to about 12, or up to about 11 amino acids.

Analysis of the crystal structure of NS5A (PDB: 1ZH1) revealed the presence of a β-hairpin in the C34-portion that is primarily responsible for interactions with Hsp70 (NS5A₁₇₁₋₁₇₉). Comparison between known sequential variants of functional proteins revealed four non-variant, hydrophobic residues in the region that, we hypothesized, form basis of molecular interactions, namely Phe₁₇₁, Val₁₇₃, Leu₁₇₅ and Tyr₁₇₈. Without being bound to a particular theory, it is believed that three of those residues form hydrophobic patch (“nucleus”) on the “functional side” of the molecule, whereas the fourth (Leu₁₇₅) may function as “locking residue” in the β-turn region.

To prepare functional, antiviral peptides derived from this region, in certain embodiments additional flanking residues that facilitate formation of a stable 13-hairpin molecule are provided. In addition, in certain embodiments (D)Pro was introduced in the position previously occupied by Gly₁₇₄ which induces a bend conformation. In certain embodiments stabilizing bridges are used to cyclize/stabilize the molecule. Thus, in certain embodiments, disulfide (—S—S—) bonds or thioether (—S—CH₂—) bridges can be included to additionally stabilize the β-hairpin structure of the molecule. To strengthen NS5A/Hsp70 hydrophobic interactions that facilitate antiviral activity, in certain embodiments, Leu₁₇₅ is substituted with Cha (L-cyclohexylalanine) which possesses higher hydrophobicity but is fairly similar to Leu. In certain embodiments similar strategies are applied to remaining residues, taking also into account void spaces(s) that are available in the original crystal structure of NS5A, which in turn can be used to accommodate specific side-chains of unusual amino acids without excessive disturbance of binding interface. One set of illustrative viable substitutions is:
for Phe₁₇₁: Dpa, ²Nal and Trp; and/or
for Val₁₇₃: Chg and Cys(tBut); and/or
for Tyr₁₇₁: Bip and Dpa.
   and the incorporation of any one or more of these substitutions in the peptides described herein is contemplated.

In certain embodiments, since the NS5A/Hsp70 interaction takes place inside a cell, in certain embodiments, additional amino acids or amino acid sequences (e.g. multiple arginine (Arg) residues and/or other cell penetrating peptides (CPPs) can be incorporated into the peptide(s).

In certain embodiments the peptide comprises the amino acid sequence according to the formula X⁴-X⁵-X⁶-X⁷-X⁸-X⁹-X¹⁰-X¹¹ (SEQ ID NO:3) or the retro, inverso, or retro-inverso form of this amino acid sequence where X⁴ is an amino acid selected from the group consisting of Phe, Nal, Dpa, and Trp, or conservative substitutions thereof, X⁵ is an amino acid selected from the group consisting of Leu, and Arg, or conservative substitutions thereof, X⁶ is an amino acid selected from the group consisting of Val, Chg, and Cys, or conservative substitutions thereof, X⁷ is an amino acid selected from the group consisting of Gly, Pro, and (D)Pro, or conservative substitutions thereof, X⁸ is an amino acid selected from the group consisting of Leu, and Cha, or conservative substitutions thereof, X⁹ is an amino acid selected from the group consisting of Asn, and His, or conservative substitutions thereof, X¹⁰ is an amino acid selected from the group consisting of Gln, Glu, and Arg, or conservative substitutions thereof, and X¹¹ is an amino acid selected from the group consisting of Tyr, Bip, and Dpa, or conservative substitutions thereof. Additional residues that can be present are selected to preserve the O-hairpin conformation and/or to improve solubility and/or to facilitate entry of the peptide into a cell.

In certain embodiments, the peptide comprises the amino acid sequence X⁴-X⁵-X⁶-X⁷-X⁸-X⁹-X¹⁰-X¹¹-X¹² (SEQ ID NO:4) or the retro, inverso, or retro-inverso form of this amino acid sequence where X¹-X¹¹ are defined as described above and X¹² is an amino acid selected from the group consisting of Leu, Pro, and Arg, or conservative substitutions thereof. In certain embodiments the peptide comprises the amino acid sequence X³-X⁴-X⁵-X⁶-X⁷-X⁸-X⁹-X¹⁰-X¹¹-X¹² (SEQ ID NO:5) or the retro, inverso, or retro-inverso form of this amino acid sequence where X⁴-X¹² are as described above and X³ is an amino acid selected from the group consisting of Thr, Ser, and Arg, or conservative substitutions thereof.

In certain embodiments the peptide comprises an amino acid sequence according to the formula X¹ₘ-X²ₙ-X³-X⁴-X⁵-X⁶-X⁷-X⁸-X⁹-X¹⁰-X¹¹-X¹²-X¹³ₒ-X¹⁴ₚ or the retro, inverso, or retro-inverso form of this amino acid sequence where X³-X¹² are as described above, m, n, o, and p are independently 0 or 1; X¹ is present or absent and when present is Arg, or a conservative substitution thereof, X² is present or absent and when present is an amino acid sequence selected from the group consisting of Val, and Cys, or conservative substitutions thereof, X¹³ is present or absent and when present is an amino acid selected from the group consisting of Val, and Cys, or conservative substitutions thereof; and X¹⁴ is present or absent and, when present is an Arg or a conservative substitution thereof. In certain embodiments X⁴ is Phe or a conservative substitution thereof, and/or X⁶ is Val or a conservative substitution thereof, and/or X⁸ Leu or a conservative substitution thereof; and/or X¹⁰ is Tyr, or a conservative substitution thereof. In certain embodiments X⁴ is selected from the group consisting of Dpa, ²Nal, and Trp; and/or X⁶ is selected from the group consisting of Chg, and Cys(tBut); and/or X¹¹ is selected from the group consisting of Bip and Dpa. In certain embodiments the amino acid sequence of the peptide comprises a sequence selected from the group consisting of Phe-Leu-Val-Gly-Leu-Asn-Gln-Tyr (SEQ ID NO:6), Phe-Arg-Val-Gly-Leu-His-Glu-Tyr (SEQ ID NO:7), Phe-Arg-Val-Gly-Leu-His-Glu-Tyr (SEQ ID NO:8), Phe-Arg-Val-(D)Pro-Cha-His-Glu-Tyr (SEQ ID NO:9), Phe-Arg-Val-(D)Pro-Cha-His-Arg-Tyr (SEQ ID NO:10), Phe-Arg-Val-(D)Pro-Cha-His-Arg-Tyr (SEQ ID NO:11), Phe-Arg-Val-(D)Pro-Cha-His-Arg-Bip (SEQ ID NO:12), Phe-Arg-Val-(D)Pro-Cha-His-Arg-Dpa (SEQ ID NO:13), Phe-Arg-Chg-(D)Pro-Cha-His-Arg-Tyr (SEQ ID NO:14), Phe-Arg-Cys^{stBu}-(D)Pro-Cha-His-Arg-Tyr (SEQ ID NO:15), ²Nal-Arg-Val-(D)Pro-Cha-His-Arg-Tyr (SEQ ID NO:16), Dpa-Arg-Val-(D)Pro-Cha-His-Arg-Tyr (SEQ ID NO:17), Trp-Arg-Val-(D)Pro-Cha-His-Arg-Dpa (SEQ ID NO:18), ²Nal-Arg-Chg-(D)Pro-Cha-His-Arg-Bip (SEQ ID NO:19), ²Nal-Arg-Val-(D)Pro-Cha-His-Arg-Tyr (SEQ ID NO:20), ²Nal-Arg-Val-(D)Pro-Cha-His-Arg-Tyr (SEQ ID NO:21), ²Nal-Arg-Val-(D)Pro-Cha-His-Arg-Tyr (SEQ ID NO:22), ²Nal-Arg-Val-(D)Pro-Cha-His-Arg-Tyr (SEQ ID NO:23), ²Nal-Arg-Chg-(D)Pro-Cha-His-Arg-Tyr (SEQ ID NO:24), ²Nal-Arg-Chg-(D)Pro-Cha-His-Arg-Tyr (SEQ ID NO:25). In any of these sequences (D)Pro can be replaced with Pro. In certain embodiments the amino acid sequence of the peptide comprises or consists of a sequence selected from the group consisting of Val-Thr-Phe-Leu-Val-Gly-Leu-Asn-Gln-Tyr-Leu-Val (SEQ ID NO:26), Val-Ser-Phe-Arg-Val-Gly-Leu-His-Glu-Tyr-Pro-Val (SEQ ID NO:27), Tyr-Phe-Val-Pro-His-Glu-Ser-Gly-Arg-Val-Val-Leu (SEQ ID NO:28), Cys-Ser-Phe-Arg-Val-Gly-Leu-His-Glu-Tyr-Pro-Cys (SEQ ID NO:29), Cys-Ser-Phe-Arg-Val-Pro-Cha-His-Glu-Tyr-Pro-Cys (SEQ ID NO:30), Arg-Cys-Arg-Phe-Arg-Val-Pro-Cha-His-Arg-Tyr-Arg-Cys-Arg (SEQ ID NO:31), Arg-Cys-Arg-Phe-Arg-Val-Pro-Cha-His-Arg-Tyr-Arg-Cys-Arg (SEQ ID NO:32), Arg-Cys-Arg-Phe-Arg-Val-Pro-Cha-His-Arg-Bip-Arg-Cys-Arg (SEQ ID NO:33), Arg-Cys-Arg-Phe-Arg-Val-Pro-Cha-His-Arg-Dpa-Arg-Cys-Arg (SEQ ID NO:34), Arg-Cys-Arg-Phe-Arg-Chg-Pro-Cha-His-Arg-Tyr-Arg-Cys-Arg (SEQ ID NO:35), Arg-Cys-Arg-Phe-Arg-Cys-Pro-Cha-His-Arg-Tyr-Arg-Cys-Arg (SEQ ID NO:36), Arg-Cys-Arg-²Nal-Arg-Val-Pro-Cha-His-Arg-Tyr-Arg-Cys-Arg (SEQ ID NO:37), Arg-Cys-Arg-Dpa-Arg-Val-Pro-Cha-His-Arg-Tyr-Arg-Cys-Arg (SEQ ID NO:38), Arg-Cys-Arg-Trp-Arg-Val-Pro-Cha-His-Arg-Dpa-Arg-Cys-Arg (SEQ ID NO:39), Arg-Cys-Arg-²Nal-Arg-Chg-Pro-Cha-His-Arg-Bip-Arg-Cys-Arg (SEQ ID NO:40), Cys-Arg-²Nal-Arg-Val-Pro-Cha-His-Arg-Tyr-Arg-Cys(SEQ ID NO:41), Cys-Arg-²Nal Nal-Arg-Val-Pro-Cha-His-Arg-Tyr-Arg-Cys (SEQ ID NO:42), Arg-²Nal-Arg-Val-Pro-Cha-His-Arg-Tyr-Arg-Cys (SEQ ID NO:43), Arg-Nal-Arg-Val-Pro-Cha-His-Arg-Tyr-Arg (SEQ ID NO:44), Cys-Arg-²Nal-Arg-Chg-Pro-Cha-His-Arg-Tyr-Arg-Cys(SEQ ID NO:45), and Arg-Nal-Arg-Chg-Pro-Cha-His-Arg-Tyr-Arg (SEQ ID NO:46), or conservative substitutions of one, two, or three residues comprising said sequences.

In certain embodiments the peptide comprises one or more “D” amino acids. In certain embodiments X⁷ comprises a (D)proline. In certain embodiments the peptide comprises all “D” amino acids.

In certain embodiments the amino acid sequence of the peptide comprises the amino acid residues of X⁴-X¹¹, or X⁴-X¹², or X³-X¹², X¹², or X²-X¹² as shown in Table 1. In certain embodiments the amino acid sequence of the peptide comprises or consists of an amino acid sequence of a peptide shown in Table 1. In certain embodiments the peptide comprises all “L” amino acids. In certain embodiments the amino acid sequence of said peptide comprises the inverse of the previously identified amino acid sequences.

In various embodiments the peptide can further comprise a cell penetrating peptide (e.g., attached to the amino or carboxyl terminus and/or complexed with the peptide). In certain embodiments the cell penetrating peptide comprises a poly Arg tag (e.g., R-(Ahx-R)₆) or a tat peptide. In certain embodiments the cell penetrating peptide comprises the amino acid sequence of a peptide shown in Table 2.

In various embodiments the peptide is cyclized, e.g., by the formation of a disulfide (—S—S—) bond or a thioether (—S—CH₂—) bridge., while in other embodiments, the peptide lacks an inter-amino acid linkage other than a peptide bond.

In various embodiments the peptide bears a protecting group on the carboxyl terminus and/or on the amino terminus and/or attached to an internal residue, or attached to a terminal residue via a side chain. Illustrative protecting groups include, but are not limited to, acetyl, amide, and 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh),Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z),2-bromobenzyloxycarbonyl (2-Br—Z), Benzyloxymethyl (Bom), t-butoxycarbonyl (Boc), cyclohexyloxy (cHxO),t-butoxymethyl (Bum), t-butoxy (tBuO), t-Butyl (tBu), Acetyl (Ac), and Trifluoroacetyl (TFA).

It will be recognized that these embodiments are intended to be illustrative and not limiting. Using the teachings provided herein other suitable β-hairpin conformation anti-viral peptides can readily be prepared by one of skill in the art.

TABLE 1
Peptides that can be used to inhibit HCV infection.

| Peptide | | | | | | | | | | | | | | | | | | |
|---|---|---|---|---|---|---|---|---|---|---|---|---|---|---|---|---|---|---|
| SEQ ID | | | | | | | | | | | | | | | | | | Cycli- |
| No | P¹ | CPP | X¹ | X² | X³ | X⁴ | X⁵ | X⁶ | X⁷ | X⁸ | X⁹ | X¹⁰ | X¹¹ | X¹² | X¹³ | X¹⁴ | P² | zation |
| Crystal | | | | Val | Thr | Phe | Leu | Val | Gly | Leu | Asn | Gln | Tyr | Leu | Val | | | |
| Structure | | | | | | | | | | | | | | | | | | |
| SEQ ID | | | | | | | | | | | | | | | | | | |
| NO: 47 | | | | | | | | | | | | | | | | | | |
| HCV1 | | | | Val | Ser | Phe | Arg | Val | Gly | Leu | His | Glu | Tyr | Pro | Val | | —NH₂ | No |
| SEQ ID | | | | | | | | | | | | | | | | | | |
| NO: 48 | | | | | | | | | | | | | | | | | | |
| HCV3 | | | | Cys^{S—} | Ser | Phe | Arg | Val | Gly | Leu | His | Glu | Tyr | Pro | Cys^{S—} | | —NH₂ | —S—S— |
| SEQ ID | | | | | | | | | | | | | | | | | | |
| NO: 50 | | | | | | | | | | | | | | | | | | |
| HCV4 | | R-(Ahx- | | Cys^{S—} | Ser | Phe | Arg | Val | (D) | Cha | His | Glu | Tyr | Pro | Cys^{S—} | | —NH₂ | —S—S— |
| SEQ ID | | R)₆-Ahx- | | | | | | | Pro | | | | | | | | | |
| NO: 51 | | Ahx- | | | | | | | | | | | | | | | | |
| HCV5 | | | Arg | Cys^{S-} | Arg | Phe | Arg | Val | (D) | Cha | His | Arg | Tyr | Arg | Cys^{S-} | Arg | —NH₂ | —S—S— |
| SEQ ID | | | | | | | | | Pro | | | | | | | | | |
| NO: 52 | | | | | | | | | | | | | | | | | | |
| HCV6 | | Pal- | Arg | Cys^{S-} | Arg | Phe | Arg | Val | (D) | Cha | His | Arg | Tyr | Arg | Cys^{S-} | Arg | —NH₂ | —S—S— |
| SEQ ID | | Ahx-Ahx- | | | | | | | Pro | | | | | | | | | |
| NO: 53 | | | | | | | | | | | | | | | | | | |
| HCV7 | | | Arg | Cys^{S-} | Arg | Phe | Arg | Val | (D) | Cha | His | Arg | Bip | Arg | Cys^{S-} | Arg | —NH₂ | —S—S— |
| SEQ ID | | | | | | | | | Pro | | | | | | | | | |
| NO: 54 | | | | | | | | | | | | | | | | | | |
| HCV8 | | | Arg | Cys^{S-} | Arg | Phe | Arg | Val | (D) | Cha | His | Arg | Dpa | Arg | Cys^{S-} | Arg | —NH₂ | —S—S— |
| SEQ ID | | | | | | | | | Pro | | | | | | | | | |
| NO: 55 | | | | | | | | | | | | | | | | | | |
| HCV9 | | | Arg | Cys^{S-} | Arg | Phe | Arg | Chg | (D) | Cha | His | Arg | Tyr | Arg | Cys^{S-} | Arg | —NH₂ | —S—S— |
| SEQ ID | | | | | | | | | Pro | | | | | | | | | |
| NO: 56 | | | | | | | | | | | | | | | | | | |
| HCV10 | | | Arg | Cys^{S-} | Arg | Phe | Arg | CystBu | (D) | Cha | His | Arg | Tyr | Arg | Cys^{S-} | Arg | —NH₂ | —S—S— |
| SEQ ID | | | | | | | | | Pro | | | | | | | | | |
| NO: 57 | | | | | | | | | | | | | | | | | | |
| HCV11 | | | Arg | Cys^{S-} | Arg | ²Nal | Arg | Val | (D) | Cha | His | Arg | Tyr | Arg | Cys^{S-} | Arg | —NH₂ | —S—S— |
| SEQ ID | | | | | | | | | Pro | | | | | | | | | |
| NO: 58 | | | | | | | | | | | | | | | | | | |
| HCV12 | | | Arg | Cys^{S-} | Arg | Dpa | Arg | Val | (D) | Cha | His | Arg | Tyr | Arg | Cys^{S-} | Arg | —NH₂ | —S—S— |
| SEQ ID | | | | | | | | | Pro | | | | | | | | | |
| NO: 59 | | | | | | | | | | | | | | | | | | |
| HCV13 | | | Arg | Cys^{S-} | Arg | Trp | Arg | Val | (D) | Cha | His | Arg | Dpa | Arg | Cys^{S-} | Arg | —NH₂ | —S—S— |
| SEQ ID | | | | | | | | | Pro | | | | | | | | | |
| NO: 60 | | | | | | | | | | | | | | | | | | |
| HCV14 | | | Arg | Cys^{S-} | Arg | ²Nal | Arg | Chg | (D) | Cha | His | Arg | Bip | Arg | Cys^{S-} | Arg | —NH₂ | —S—S— |
| SEQ ID | | | | | | | | | Pro | | | | | | | | | |
| NO: 61 | | | | | | | | | | | | | | | | | | |
| HCV15 | | | | Cys^{S-} | Arg | ²Nal | Arg | Val | (D) | Cha | His | Arg | Tyr | Arg | Cys^{S-} | | —NH₂ | —S—S— |
| SEQ ID | | | | | | | | | Pro | | | | | | | | | |
| NO: 62 | | | | | | | | | | | | | | | | | | |
| HCV15R | | | | Cys^{SH} | Arg | ²Nal | Arg | Val | (D) | Cha | His | Arg | Tyr | Arg | Cys^{SH} | | —NH₂ | No/ |
| SEQ ID | | | | | | | | | Pro | | | | | | | | | reduced |
| NO: 63 | | | | | | | | | | | | | | | | | | form |
| HCV16 | Ac | | | | Arg | ²Nal | Arg | Val | (D) | Cha | His | Arg | Tyr | Arg | Cys^{S-} | | —NH₂ | —S—CH₂—/ |
| SEQ ID | | | | | | | | | Pro | | | | | | | | | Thioether |
| NO: 64 | | | | | | | | | | | | | | | | | | |
| HCV17 | Ac | | | | Arg | ²Nal | Arg | Val | (D) | Cha | His | Arg | Tyr | Arg | | | NH— | —S—CH₂—/ |
| SEQ ID | | | | | | | | | Pro | | | | | | | | CH₂CH₂ | Thioether |
| NO: 65 | | | | | | | | | | | | | | | | | CH₂CH₂ | |
| | | | | | | | | | | | | | | | | | S— | |
| HCV18 | | | | Cys | Arg | ²Nal | Arg | Chg | (D) | Cha | His | Arg | Tyr | Arg | Cys | | —NH₂ | —S—S— |
| SEQ ID | | | | | | | | | Pro | | | | | | | | | |
| NO: 66 | | | | | | | | | | | | | | | | | | |
| HCV19 | Ac | | | | Arg | ²Nal | Arg | Chg | (D) | Cha | His | Arg | Tyr | Arg | | | NH— | —S—CH₂—/ |
| SEQ ID | | | | | | | | | Pro | | | | | | | | CH₂CH₂ | Thioether |
| NO: 67 | | | | | | | | | | | | | | | | | CH₂CH₂ | |
| | | | | | | | | | | | | | | | | | S— | |
| HCV18R1 | | | | (D)Cys | (D) | (D)Tyr | (D) | (D)His | (D) | (L) | (D) | (D) | (D)² | (D) | (D) | | —NH₂ | |
| SEQ ID | | | | | Arg | | Arg | | Cha | Pro | Chg | Arg | Nal | Arg | Cys | | | |
| NO: 68 | | | | | | | | | | | | | | | | | | |

HCV2 (shown in FIG. 9) is a scrambled sequence.
P1 is an optional first protecting group, P2, as shown, is an optional second (carboxyl terminal) protecting group or a linker for cyclizing the peptide.
Cyclization —S—S— = disulfide bridge.
(Poly Arg tag: R-(Ahx-R)₆—CONH₂).
Abbreviations: Cha-(L) Cyclohexylalanine, Tle-(L)-tert-Leucine, Chg-(L)-Cyclohexylglycine, PheF5-Pentafluoro-(L)-phenylalanine, Dpa- 3,3-Diphenyl-(L)-alanine, Bip-(L)-Biphenylalanine, 1Nal-3-(1-Naphthyl)-(L)-alanine, 2Nal-3-(2-Naphthyl)-(L)-alanine, Ahx-6-Aminohexanoic acid, Pal-Palmitic acid.

TABLE 2
Illustrative cell penetrating peptides (CPPs).

| | | | SEQ ID |
|---|---|---|---|
| Classes | Peptides | Sequences | NO |
| Basic | Tat and related peptides | | |
| | HIV-1 Tat (48-60) Rn | GRKKRRQRRRPPQ | 69 |
| | (n = ~7-11) | | |
| | R9-Tat | GRRRRRRRRRPPQ | 70 |
| | Tatp | YGRKKRRQRRR | 71 |
| | Arginine-rich peptides | | |
| | Arg8 | RRRRRRRR | 72 |
| | Arg6 | RRRRRR | 73 |
| | pVEC | LLIILRRRIRKQAHAHSK⁶³⁵ | 74 |
| | HIV-1 Rev-(34-50) | TRQARRNRRRRWRERQR | 75 |
| | R7W | RRRRRRRW-NH2 | 76 |
| | TatP59W | GRKKRRQRRRPWQ | 77 |
| | FHV Coat-(35-49) | RRRRNRTRRNRRRVR | 78 |
| | BMV Gag-(7-25) | KMTRAQRRAAARRNRWTAR | 79 |
| | HTLV-II Rex-(4-16) | TRRQRTRRARRNR | 80 |
| | CCMV Gag-(7-25) | KLTRAQRRAAARKNKRNTR | 81 |
| | P22 N-(14-30) | NAKTRRHERRRKLAIER | 82 |
| Basic/ | Antennapedia (43-58) | RQIKIWFQNRRMKWKK | 83 |
| amphiphilic | (penetratin) | | |
| | Fluoro-Penetratin | Fluo-RQIKIWFQNRRMKWKK-NH2 | 84 |
| | Pen2W2F | Fluo-RQIKIFFQNRRMKFKK-NH2 | 85 |
| | model amphipathic peptide | KLALKLALKALKAALKLA-NH2 | 86 |
| | PenArg | Fluo-RQIRIWFQNRRMRWRR-NH2 | 87 |
| | PenLys | Fluo-KQIKIWFQNKKMKWKK-NH2 | 88 |
| | E N-(1-22) | MDAQTRRRERRAEKQAQWKAAN | 89 |
| | B 21 N-(12-29) | TAKTRYKARRAELIAERR | 90 |
| | Yeast PRP6-(129-144) | TRRNKRNRIQEQLNRK | 91 |
| | Hum U2AF-(142-153) | SQMTRQARRLYV | 92 |
| Chimera | transportan | GWTLNSAGYLLGKINLKALAALAKK | 93 |
| | (galanin/mastoparan) | IL | |
| | Transportan-10 | AGYLLGKINLKALAALAKKIL | 94 |
| | MAP | KLALKLALKALKAALKLA | 95 |
| | KALA | WEAKLAKALAKALAKHLAKALAK | 96 |
| | | ALKACEA | |
| | ppTG1 | GLFKALLKLLKSLWKLLLKA | 97 |
| | Pep-1 (hydrophobic/NLS) | KETWWETWWTEWSQPKKKRKV- | 98 |
| | | cysteamine | |
| | MPG | GALFLGFLGAAGSTMGAWSQPKSK | 99 |
| | | RKV | |
| Hydrophobic | membrane | AAVALLPAVLLALLP | 100 |
| | translocating sequence | | |
| | peptide(MTS) | | |

### Formulations and Administration.

Pharmaceutical Formulations.

In certain embodiments, one or more antiviral peptides described herein are administered to a mammal in need thereof, to a cell, to a tissue, and the like to inhibit intracellular production/propagation of a positive sense RNA virus that has an internal ribosome entry site (e.g., Hepatitis C, Hepatitis A, etc.) and/or to inhibit NS5A-mediated IRES activity.

These active agents (antiviral peptides) can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, the disulfide salts of a number of delivery agents are described in PCT Publication WO 2000/059863 which is incorporated herein by reference. Similarly, acid salts of therapeutic peptides, peptoids, or other mimetics, and can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

In various embodiments preparation of esters typically involves functionalization of hydroxyl and/or carboxyl groups that are present within the molecular structure of the active agent (e.g., antiviral peptide). In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agents (antiviral peptides) identified herein are useful for parenteral, topical, oral, nasal (or otherwise inhaled), rectal, or local administration, such as by aerosol or transdermally, for prophylactic and/or therapeutic treatment of infection (e.g., Hepatitis C infection). The compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, liposomes, etc.

The active agents (e.g., antiviral peptide(s)) described herein can also be combined with a pharmaceutically acceptable carrier (excipient) to form a pharmacological composition. In certain embodiments, pharmaceutically acceptable carriers include those approved by a regulatory agency of the Federal or a state government or listed in the U.S. Pharmacopeia or other generally recognized pharmacopeia for use in/on animals, and more particularly in/on humans. A “carrier” refers to, for example, a diluent, adjuvant, excipient, auxiliary agent or vehicle with which an active agent of the present invention is administered.

Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., active peptide) and the resulting composition is compressed. Where necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

It is contemplated that in certain embodiments, the antiviral peptides described herein can be delivered orally or via injection (or via other routes) without particular protective carriers. Nevertheless, in certain embodiments, peptide delivery can be enhanced by the use of protective excipients. This is typically accomplished either by complexing the polypeptide with a composition to render it resistant to acidic and enzymatic hydrolysis and/or from clearance in the blood stream, or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

In certain embodiments elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy (1998) Biotechnol. Prog. 14: 108; Johnson et al. (1996), Nature Med. 2: 795; Herbert et al. (1998), Pharmaceut. Res. 15, 357) a dry powder composed of biodegradable polymeric microspheres containing the protein in a polymer matrix that can be compounded as a dry formulation with or without other agents.

The ProLease microsphere fabrication process was specifically designed to achieve a high protein encapsulation efficiency while maintaining protein integrity. The process consists of (i) preparation of freeze-dried protein particles from bulk protein by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the protein, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the protein is maintained in the solid state in the absence of water, thus minimizing water-induced conformational mobility of the protein, preventing protein degradation reactions that include water as a reactant, and avoiding organic-aqueous interfaces where proteins may undergo denaturation. A preferred process uses solvents in which most proteins are insoluble, thus yielding high encapsulation efficiencies (e.g., greater than 95%).

In certain embodiments the peptide(s) are formulated with a non-covalent carrier. Non-covalent carriers are well known to those of skill and include, for example the Chariot reagent by Active Motif which is based on a short synthetic signaling peptide called Pep-1 (see, e.g., Loudet et al. (2008) Org. Biomol. Chem. 6(24): 4516-4522). Other non-covalent carriers include, but are not limited to R(8), and azo-R(8) (Id.).

In certain embodiments, the peptide(s) are complexed with lipids or formulated in liposomes. Methods of producing liposomes and complexing or encapsulating compounds therein are well known to those of skill in the art (see, e.g., Debs and Zhu (1993) WO 93/24640; Mannino and Gould-Fogerite (1988) BioTechniques 6(7): 682-691; Rose U.S. Pat. No. 5,279,833; Brigham (1991) WO 91/06309; and Felgner et al. (1987) Proc. Natl. Acad. Sci. USA 84: 7413-7414, and the like).

In certain embodiments the peptide(s) are formulated in a “vault” nanocapsule. Vaults are self-assembled ribonucleoprotein nanocapsules that consist of multiple copies of three proteins (major vault protein, VPARP, and TEP1) and an untranslated RNA. Vaults have been modified to produce nanocapsules capable of delivering therapeutic moieties (e.g., small organic molecules, proteins, etc.). Such uses of vault nanocapsules are described, for example, by Kar et al. (2011) PLoS One, 6: e18758).

In certain embodiments the antiviral peptide(s) described herein are formulated in a nanoemulsion. Nanoemulsions include, but are not limited to oil in water (O/W) nanoemulsions, and water in oil (W/O) nanoemulsions. Nanoemulsions can be defined as emulsions with mean droplet diameters ranging from about 20 to about 1000 nm. Usually, the average droplet size is between about 20 nm or 50 nm and about 500 nm. The terms sub-micron emulsion (SME) and mini-emulsion are used as synonyms.

Illustrative oil in water (O/W) and/or (W/O) nanoemulsions include, but are not limited to:

1) Surfactant micelles—micelles composed of small molecules surfactants or detergents (e.g., SDS/PBS/2-propanol) which are suitable for predominantly hydrophobic peptides;

2) Polymer micelles—micelles composed of polymer, copolymer, or block copolymer surfactants (e.g., Pluronic L64/PBS/2-propanol) which are suitable for predominantly hydrophobic peptides;

3) Blended micelles: micelles in which there is more than one surfactant component or in which one of the liquid phases (generally an alcohol or fatty acid compound) participates in the formation of the micelle (e.g., Octanoic acid/PBS/EtOH) which are suitable for predominantly hydrophobic peptides;

4) Integral peptide micelles—blended micelles in which the peptide serves as an auxiliary surfactant, forming an integral part of the micelle (e.g., amphipathic peptide/PBS/mineral oil) which are suitable for amphipathic peptides; and

5) Pickering (solid phase) emulsions—emulsions in which the peptides are associated with the exterior of a solid nanoparticle (e.g., polystyrene nanoparticles/PBS/no oil phase) which are suitable for amphipathic peptides.

As indicated above, in certain embodiments the nanoemulsions comprise one or more surfactants or detergents. In some embodiments the surfactant is a non-anionic detergent (e.g., a polysorbate surfactant, a polyoxyethylene ether, etc.). Surfactants that find use in the present invention include, but are not limited to surfactants such as the TWEEN®, TRITON®, and TYLOXAPOL® families of compounds.

In certain embodiments the emulsions further comprise one or more cationic halogen containing compounds, including but not limited to, cetylpyridinium chloride. In still further embodiments, the compositions further comprise one or more compounds that increase the interaction (“interaction enhancers”) of the composition with microorganisms (e.g., chelating agents like ethylenediaminetetraacetic acid, or ethylenebis(oxyethylenenitrilo)tetraacetic acid in a buffer).

In some embodiments, the nanoemulsion further comprises an emulsifying agent to aid in the formation of the emulsion. Emulsifying agents include compounds that aggregate at the oil/water interface to form a kind of continuous membrane that prevents direct contact between two adjacent droplets. Certain embodiments of the present invention feature oil-in-water emulsion compositions that may readily be diluted with water to a desired concentration without impairing their anti-pathogenic properties.

In addition to discrete oil droplets dispersed in an aqueous phase, certain oil-in-water emulsions can also contain other lipid structures, such as small lipid vesicles (e.g., lipid spheres that often consist of several substantially concentric lipid bilayers separated from each other by layers of aqueous phase), micelles (e.g., amphiphilic molecules in small clusters of 50-200 molecules arranged so that the polar head groups face outward toward the aqueous phase and the apolar tails are sequestered inward away from the aqueous phase), or lamellar phases (lipid dispersions in which each particle consists of parallel amphiphilic bilayers separated by thin films of water).

These lipid structures are formed as a result of hydrophobic forces that drive apolar residues (e.g., long hydrocarbon chains) away from water. The above lipid preparations can generally be described as surfactant lipid preparations (SLPs). SLPs are minimally toxic to mucous membranes and are believed to be metabolized within the small intestine (see e.g., Hamouda et al., (1998) J. Infect. Disease 180: 1939).

In certain embodiments the emulsion comprises a discontinuous oil phase distributed in an aqueous phase, a first component comprising an alcohol and/or glycerol, and a second component comprising a surfactant or a halogen-containing compound. The aqueous phase can comprise any type of aqueous phase including, but not limited to, water (e.g., dionized water, distilled water, tap water) and solutions (e.g., phosphate buffered saline solution, or other buffer systems). The oil phase can comprise any type of oil including, but not limited to, plant oils (e.g., soybean oil, avocado oil, flaxseed oil, coconut oil, cottonseed oil, squalene oil, olive oil, canola oil, corn oil, rapeseed oil, safflower oil, and sunflower oil), animal oils (e.g., fish oil), flavor oil, water insoluble vitamins, mineral oil, and motor oil. In certain embodiments, the oil phase comprises 30-90 vol % of the oil-in-water emulsion (i.e., constitutes 30-90% of the total volume of the final emulsion), more preferably 50-80%.

In certain embodiments the alcohol, when present, is ethanol.

While the present invention is not limited by the nature of the surfactant, in some preferred embodiments, the surfactant is a polysorbate surfactant (e.g., TWEEN 20®, TWEEN 40®, TWEEN 60®, and TWEEN 80®), a pheoxypolyethoxyethanol (e.g., TRITON® X-100, X-301, X-165, X-102, and X-200, and TYLOXAPOL®), or sodium dodecyl sulfate, and the like.

In certain embodiments a halogen-containing component is present. the nature of the halogen-containing compound, in some preferred embodiments the halogen-containing compound comprises a chloride salt (e.g., NaCl, KCl, etc.), a cetylpyridinium halide, a cetyltrimethylammonium halide, a cetyldimethylethylammonium halide, a cetyldimethylbenzylammonium halide, a cetyltributylphosphonium halide, dodecyltrimethylammonium halides, tetradecyltrimethylammonium halides, cetylpyridinium chloride, cetyltrimethylammonium chloride, cetylbenzyldimethylammonium chloride, cetylpyridinium bromide, cetyltrimethylammonium bromide, cetyldimethylethylammonium bromide, cetyltributylphosphonium bromide, dodecyltrimethylammonium bromide, tetradecyltrimethylammonium bromide, and the like

In certain embodiments the emulsion comprises a quaternary ammonium compound. Quaternary ammonium compounds include, but are not limited to, N-alkyldimethyl benzyl ammonium saccharinate, 1,3,5-Triazine-1,3,5(2H,4H,6H)-triethanol; 1-Decanaminium, N-decyl-N,N-dimethyl-, chloride (or) Didecyl dimethyl ammonium chloride; 2-(2-(p-(Diisobuyl)cresosxy)ethoxy)ethyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl)benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl) octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethylbenzyl ammonium chloride; and n-octadecyl dimethyl benzyl ammonium chloride.

Nanoemulsion formulations and methods of making such are well known to those of skill in the art and described for example in U.S. Pat. Nos. 7,476,393, 7,468,402, 7,314,624, 6,998,426, 6,902,737, 6,689,371, 6,541,018, 6,464,990, 6,461,625, 6,419,946, 6,413,527, 6,375,960, 6,335,022, 6,274,150, 6,120,778, 6,039,936, 5,925,341, 5,753,241, 5,698,219, and 5,152,923 and in Fanun et al. (2009) Microemulsions: Properties and Applications (Surfactant Science), CRC Press, Boca Ratan Fl.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

The foregoing formulations are intended to be illustrative and not limiting. Using the teachings provided herein, other suitable formulations will be available to one of skill in the art.

Administration

In certain therapeutic applications, the antiviral peptide(s) described herein are administered, e.g., orally, via injection, via inhalation, etc. topically administered or administered to the oral or nasal cavity, to a patient suffering from a viral infection infection (e.g., hepatitis C) or at risk for infection in an amount sufficient to prevent and/or cure and/or at least partially prevent or arrest the disease and/or its complications. Amounts effective for this use will depend upon the severity of the disease (infection), the general state of the patient's health, and other active agent(s) (e.g., interferon) that may or may not be included in the therapeutic regimen. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms in and/or reduce infectivity and/or propagation of the virus) in the patient.

The concentration of active agent(s) can vary widely, and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic and/or phophylactic regimen in a particular subject or group of subjects.

In certain embodiments the active agents of this invention are administered systemically (e.g., orally, or as an injectable) in accordance with standard methods well known to those of skill in the art. In other preferred embodiments, the agents (antiviral peptide(s)), can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

In certain embodiments the agents (antiviral peptide(s)) may be delivered topically. Other formulations for topical delivery include, but are not limited to, ointments, gels, sprays, fluids, and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

In certain embodiments, various buccal, and sublingual formulations are also contemplated.

In certain embodiments, one or more active agents (antiviral peptides) of the present invention can be provided as a “concentrate”, e.g., in a storage container (e.g., in a premeasured volume) ready for dilution, or in a soluble capsule ready for addition to a volume of water, alcohol, hydrogen peroxide, or other diluent.

While the invention is described above with respect to use in humans, it is also suitable for animal, e.g., veterinary use. Thus certain preferred organisms include, but are not limited to humans, non-human primates, canines, equines, felines, porcines, ungulates, largomorphs, and the like.

### Kits.

In another embodiment this kits are provided for the inhibition of a viral infection and/or for the treatment and/or prevention of a viral infection in a mammal. The kits typically comprise a container containing one or more of the active agents (i.e., the antiviral peptide(s) described herein. In certain embodiments the active agent(s) can be provided in a unit dosage formulation (e.g., tablet, caplet, patch, etc.) and/or may be optionally combined with one or more pharmaceutically acceptable excipients.

In addition, the kits optionally include labeling and/or instructional materials providing directions (i.e., protocols) for the practice of the methods or use of the “therapeutics” or “prophylactics” described herein. Thus, for example, the kit may contain directions for the use of the peptide(s) contained therein in the treatment of hepatitis C (or other viral infections). The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### A Cell-Permeable Hairpin Peptide Inhibits Hepatitis C Viral Nonstructural Protein 5A Mediated Translation and Virus Production

NS5A is a key regulator of hepatitis C virus (HCV) life cycle including RNA replication, assembly, and translation. We and others have shown NS5A to augment HCV IRESmediated translation. Further, Quercetin treatment and heat shock protein (HSP) 70 knockdown inhibit NS5A-driven augmentation of IRES-mediated translation and infectious virus production. We have also co-immunoprecipitated HSP70 with NS5A and demonstrated cellular colocalization leading to the hypothesis that the NS5A/HSP70 complex formation is important for IRES-mediated translation. This example describes the identification of the NS5A region responsible for complex formation through in vitro deletion analyses. Deletion of NS5A domains II and III failed to reduce HSP70 binding, whereas domain I deletion eliminated complex formation. NS5A domain I alone also bound HSP70. Deletion mapping of domain I identified the C-terminal 34 amino acids (C34) to be the interaction site. Further, addition of C34 viral protein levels, in contrast to same size control peptides from other to domains II and III restored complex formation. C34 expression significantly reduced intracellular NS5A domains. C34 also competitively inhibited NS5A-augmented IRES-mediated translation, while controls did not. Triple-alanine scan mutagenesis identified an exposed beta-sheet hairpin in C34 to be primarily responsible for NS5A-augmented IRES-mediated translation. Moreover, treatment with a 10 amino acid peptide derivative of C34 suppressed NS5A-augmented IRES-mediated translation and significantly inhibited intracellular viral protein synthesis, with no associated cytotoxicity. These results indicate that the NS5A/HSP70 complex augments viral IRES-mediated translation, identify a sequence-specific hairpin element in NS5A responsible for complex formation, and demonstrate the functional significance of C34 hairpin-mediated NS5A/HSP70 interaction. Identification of this element allows for further interrogation of NS5A-mediated IRES activity, sequence specific HSP recognition, and rational drug design.

### Experimental Procedures

Plasmid Constructs and Cloning.

HSP70 and HSP40 were amplified from Huh-7 cDNA using polymerase chain reaction (PCR), and NS5A was amplified from pMSCVNS5AFLAG described previously (Gonzalez et al. (2009) Hepatology50: 1756-1764). pGEX-6P-2 plasmid (Amersham, 28-9546-50) was used to generate N-terminal GST fusion HSP70, HSP40, and NS5A as well as HSP70 nucleotide binding domain and substrate binding domain. pET-28b plasmid (Novagen, 69865-3) was used to generate C-terminal His6-tagged HSP70, HSP40, and NS5A. pTRACER-EF/Bsd A plasmid (Invitrogen, V889-01) was used to generate C-terminal V5-epitope-tagged NS5A, HSP70, and HSP40 and all NS5A subclones. The C34 construct as well as NS5A domain II and domain III peptides, NS3 peptide (P19), and C34 mutagenesis constructs (ASMs) were cloned into pCCL-CMVIRES-EGFP plasmid. pRSET A plasmid (Invitrogen, V351-20) was used for generating C34 alanine mutants by site-directed mutagenesis as its smaller size compared with pCCL-CMV-IRES-EGFP allows for much more efficient mutagenesis. Two additional mutagenesis constructs were designed for ASM10 and ASM11 as alanine mutagenesis did not result in significant changes. Therefore, VAV and LTSM were mutated to SSS (M10) and SAGS (M11) for ASM10 and ASMI 1, respectively. All 13 mutagenized constructs were cloned into pCCL-CMV-IRES-EGFP. The HCV IRES reporter plasmid pNRLFC, and the GFP retroviral expression vector pMSCVGFP have been previously described (Id.).

Cell Culture.

Cell lines 293T, Huh-7, and Huh-7.5 were maintained in a humidified atmosphere containing 5% CO₂ at 37° C. in Dulbecco's modified Eagle's medium (Mediatech, 10-013-CM) supplemented with 10% fetal bovine serum (Omega Scientific, FB-01) and 2 mM L-glutamine (Invitrogen, 25030).

Infectious virus Production.

pNRLFC was in vitro transcribed, and the purified RNA was electroporated into Huh-7.5 cells to generate infectious viral supernatant as previously described (Arumugaswami et al. (2008) PLoS Pathogens, 4: e1000182). Viral assays were done using the HCV reporter virus as described previously (Gonzalez et al. (2009) Hepatology50: 1756-1764).

Antibodies.

HSP70 (Santa Cruz Biotech, C92F3A-5), HSP40 (Abcam, ab56589), NS5A (Abeam, ab20342), and V5 epitope (Millipore, AB3792).

Production and Purification of Recombinant Proteins.

GST-fusion proteins were expressed in bacteria and purified as previously described (French et al. (2007) Cancer Letts., 248: 198-210). His6-tagged proteins were expressed and purified using Ni-NTA Agarose bead slurry (Qiagen, 1018244) according to manufacturer's instructions. V5-epitope-tagged proteins were expressed using TNT T7 Coupled Reticulocyte Lysate System (Promega, L4610).

GST Pull-Down Assays.

GST pull-down assays were performed as previously described (French et al. (2007) Cancer Letts., 248: 198-210). Reactions were evaluated by western analysis using V5-epitope-tagged target proteins/peptides.

IRES Reporter Assay.

293T cells were transfected with HCV IRES reporter plasmid; either pMSCV5AFLAG or pMSCVGFP; and pCCL-CMV-IRES-EGFP plasmid expressing one of the following peptides: C34, NS5A domain II peptide, NS5A domain III peptide, NS3 peptide, or one of the C34 mutagenesis peptides. All transfections were done using Fugene 6 (Roche, 11814443001). 48 hours post transfection, Renilla and Firefly luciferase activity was determined using Dual Luciferase Assay System (Promega, E1910).

Peptide Synthesis and Characterization.

Peptides were synthesized by the solid phase method using CEM Liberty automatic microwave peptide synthesizer (CEM Corporation), applying 9-fluorenylmethyloxycarbonyl (Fmoc) chemistry (Fields et al. (1990) int. J. Pept. Protein Res., 35: 161-214) and standard, commercially available amino acid derivatives and reagents (EMD Biosciences and Chem-Impex International) Rink Amide MBHA resin (EMD Biosciences) was used as a solid support. Peptides were cleaved from resin using modified reagent K (TFA 94% (v/v); phenol, 2% (w/v); water, 2% (v/v); TIS, 1% (v/v); EDT, 1% (v/v); 2 hours) and precipitated by addition of ice-cold diethyl ether. Reduced peptides were purified by preparative reverse-phase high performance liquid chromatography (RP-HPLC) to >95% homogeneity and their purity evaluated by matrix-assisted laser desorption ionization spectrometry (MALDI-MS) as well as by analytical RP-HPLC.

Disulfide Bond Formation.

Peptides were dissolved at a final concentration of 0.25 mg/ml in 50% DMSO:H₂O and stirred overnight at room temperature. Subsequently peptides were lyophilized and re-purified on a preparative C 18 SymmetryShield™ RP-HPLC column to >95% homogeneity. Their purity was evaluated by MALDI-MS as well as by analytical RP-HPLC.

Analytical RP-HPLC:

Analytical RP-HPLC was performed on a Varian ProStar 210 HPLC system equipped with ProStar 325 Dual Wavelength UV-Vis detector with wavelengths set at 220 nm and 280 nm (Varian Inc.). Mobile phases consisted of solvent A, 0.1% TFA in water, and solvent B, 0.1% TFA in acetonitrile. Analyses of peptides were performed with an analytical reversed-phase C18 SymmetryShield™ RP18 column, 4.6×250 mm, 5 pm (Waters Corp.) applying linear gradient of solvent B from 0 to 100% over 100 min (flow rate: 1 ml/min). The HCV4 sequence (including its arginine tag) is as follows: Arg-(Ahx-Arg)₆-Ahx-Ahx-Cys-Ser-Phe-Arg-Val-(D)Pro-Cha-His-Glu-Tyr-Pro-Cys (SEQ ID NO:101), where Ahx and Cha refer to 6-aminohexanoic acid and cyclohexylalanine, respectively.

Cell Viability.

Cell viability was determined using MTT Cell Proliferation assay (ATCC, 30-1010K).

Crystal Structure Analysis.

Figures representing the crystal structure of NS5A domain I and the C34 region were generated by the PyMol software and ‘1ZH1’ pdb file of dimeric NS5A domain I reported previously (Tellinghuisen et al. (2005) Nature, 435: 374-379).

Protein Hydrophobicity Analysis.

Hydrophobicity plot of NS5A was generated using the CLC Protein Workbench software by CLC Bio. The Kyte and Doolittle scale was used for all plots with a window size of seven.

Statistical Analysis.

Error bars reflect standard deviation. P values were determined by student t test.

### Results

The C-terminal Region of NS5A Domain I is Necessary and Sufficient for its Interaction with HSP70.

We have previously shown that NS5A forms a complex with HSP70 (encoded by HSPA1A) and HSP40 (encoded by DNAJ2) (Gonzalez et al. (2009) Hepatology50: 1756-1764). To determine whether NS5A interacts directly with either or both of these HSPs, GST-fusion protein pull-down assays were performed. GST-HSP70 interacted with NS5A but GST-HSP40 showed minimal interaction (FIG. 2, panel A). NS5A and HSP70 binding was further confirmed by using GST-NS5A fusion protein as bait to successfully pull down HSP70 (FIG. 2, panel B).

NS5A consists of four domains: membrane-anchoring domain, domain I, domain II, and domain III (FIG. 4, panel B). To determine the individual domain(s) that interact(s) with HSP70, each domain was specifically deleted, and the resulting proteins were expressed through in vitro coupled transcription/translation reactions. Each deletion mutant was then tested for HSP70 binding using GST pull-down assays. Deletion of NS5A domain I abolished its interaction with HSP70, while the other deletion mutants maintained this interaction (FIG. 3, panels A and B). Furthermore, NS5A domain I, II, and III were individually expressed and tested for HSP70 binding. Only domain I of NS5A bound with HSP70, and no interaction was detected for domains II and III (FIG. 3, panel C).

To determine which region of NS5A domain I binds HSP70, N-terminal and C-terminal deletions of domain I (FIG. 4, panel C) were expressed in vitro and tested for HSP70 interaction using GST pull-down assays. Deletions of 34 and 68 amino acids at the N terminus of NS5A domain I did not have any effect on its HSP70 binding (FIG. 3, panel D). However, deletion of 34 amino acids from the C terminus abolished the HSP70-binding capability of NS5A domain I (FIG. 3, panel E). Deleting an additional 34 amino acids at the C terminus showed the same result (FIG. 3, panel E).

To demonstrate that the C-terminal region of NS5A domain I is indeed responsible for HSP70 complex formation, the C-terminal 34 amino acids of domain I were added to the NS5A domain I deletion mutant (FIG. 4, panel C). Subsequent GST pull-down assays revealed that addition of the C-terminal 34 amino acids of domain I to the NS5A domain I deletion construct restored HSP70-binding capability (FIG. 3, panel F). Results of all HSP70 binding assays are summarized in FIG. 4, panel C.

NS5A directly interacts with the nucleotide binding domain of HSP70.

HSP70 binds in a non-specific and sequence-independent manner to hydrophobic peptides through its C-terminal substrate binding domain whereas its N-terminal nucleotide-binding domain (NBD) interacts with regulatory proteins (Wegele et al. (2004) Rev. Physiol., Biochem., and Pharmacol., 151: 1-44). GST-fusion HSP70-NBD and HSP70-SBD were tested for interaction with NS5A domain I. Only HSP70-NBD interacted with domain I of NS5A (FIG. 2, panel C).

The C-Terminal 34 Amino Acid Peptide of NS5A Domain I Suppresses NS5A-Augmented IRES-mediated Translation.

After identifying the C-terminal region of NS5A as the HSP70 binding site in vitro, we sought to determine whether this region has any functional significance. NS5A has been reported to increase IRES-mediated translation in a cell culture-based bicistronic reporter system (He et al. (2003) J. General Virol., 84: 535-543) (FIG. 5A). This reporter system consists of the Firefly luciferase open reading frame (ORF) driven by HCV IRES and Renilla luciferase ORF under the control of a 5′ cap (FIG. 5A). The ratio of Firefly to Renilla luciferase activity can be used to measure the levels of IRES-mediated translation. The C-terminal 34 amino acid peptide corresponding to amino acids 165-198 of NSSA domain I (hereafter referred to as C34) was tested for its effect on altering IRES-mediated translation. (C34 corresponds to amino acids 169-202 of the NS5A variant used in the crystal structure of NS5A domain I reported previously Tellinghuisen et al. (2005) Nature, 435: 374-379); however, the crystal structure itself terminates at amino acid 198 and, therefore, includes the first 30 amino acids of C34 (FIG. 6A)). Peptides of identical length from NS5A domains II and III and a 19 amino acid peptide (P19) from NS3 (Gozdek et al. (2008) Antimicrb. Agents. and Chemotherap., 52: 393-401) were also tested. C34 was found to block NS5A-augmented IRES-mediated translation in cell culture, while NSSA domain II and III peptides and the NS3 peptide did not have any significant effects on IRES-mediated translation (FIG. 5B).

C34 Blocks Intracellular Viral Protein Synthesis.

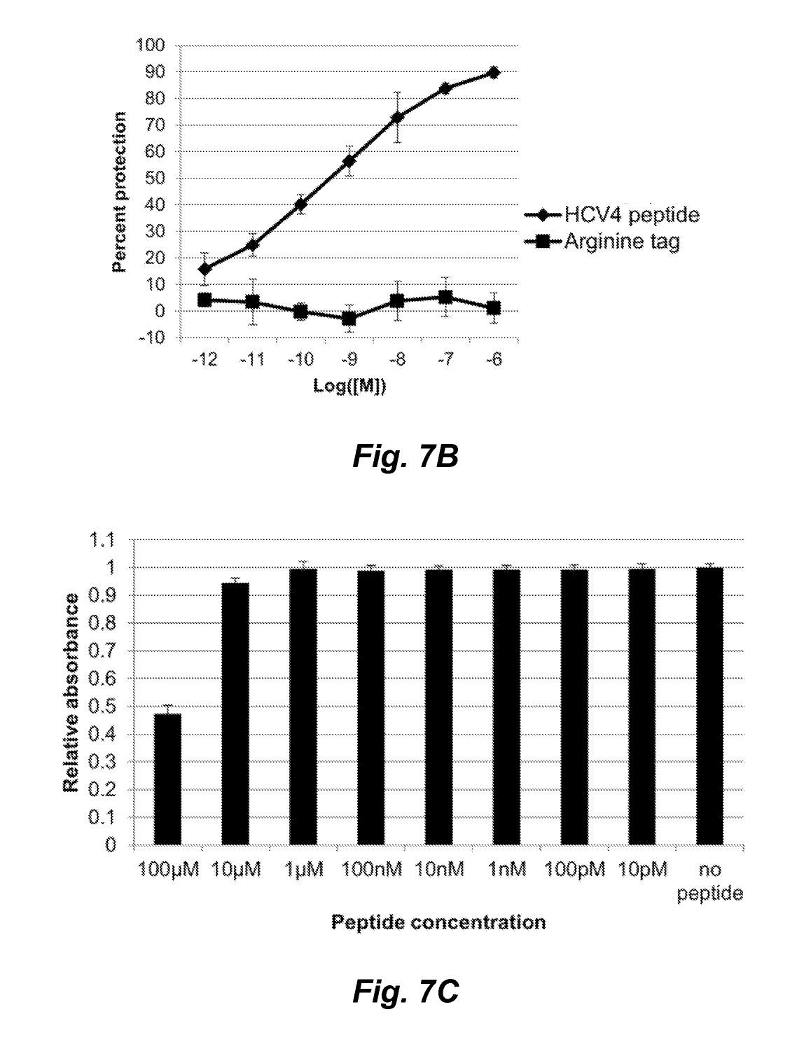

Expression of C34 in the HCV cell culture (HCVcc) system significantly reduced intracellular viral protein production as measured by Renilla luciferase reporter activity (FIG. 7A). In addition, the P19 peptide from NS3 showed modest reduction in intracellular virus in agreement with previous studies (Id.) (FIG. 7A). In contrast, expression of equal sized peptides from other areas of NS5A failed to have an effect on infection (FIG. 7A). C34 expression had no effect on cell viability as measured by MTT assay (data not shown).

Mutation of an Exposed Antiparallel Beta-Sheet Hairpin in C34 Inhibits NS5A Augmentation of IRES-Mediated Translation.

Scanning triple alanine substitution mutants of C34 were generated to determine the amino acids important for NS5A/HSP70 complex-mediated IRES translation. The IRES reporter, NS5A, and C34 mutants and wild type were transfected into cells, and IRES activity was measured. The most significant mutations affecting NSSA activity localized to amino acids 171-179 (FIG. 5C), which correspond to amino acids 175-183 of the NS5A isoform used to generate the crystal structure of NSSA domain I (Tellinghuisen et al. (2005) Nature, 435: 374-379). This region maps to an exposed anti-parallel beta-sheet hairpin based on the crystal structure (FIGS. 6A and 6B) external to the claw-like structure of NS5A domain I dimer that binds single-stranded RNA (Id.).

A 10 Amino Acid Peptide Corresponding to the C34 Hairpin Blocks Intracellular Viral Production and NS5A-Augmented IRES-Mediated Translation.

The crystal structure of NS5A domain I reveals the hairpin region to be the only moiety in C34 with secondary structure (Id.). To determine whether the hairpin alone (as opposed to the entire C34) would be sufficient for antiviral activity, a modified peptide of 10 amino acid length corresponding to the C34 hairpin structure was generated (HCV4 peptide). Two cysteine residues were added to each end to allow for disulfide bond formation and to achieve a conformation similar to the C34 hairpin. Also an arginine tag was added to the N-terminal cysteine to allow for efficient uptake by cells without any transfection reagent. Huh-7.5 cells were treated with concentrations of HCV4 peptide ranging from 100 μM to 10 pM in the HCVcc system and were immediately infected with the HCV Renilla reporter virus. The HCV4 peptide dramatically suppressed intracellular viral protein production even in picomolar doses, with an estimated IC50 of 500 pM, while the arginine tag control peptide by itself did not show any effect (FIG. 7B). MTT assays demonstrated toxicity at 100 μM and slight toxicity at 10 μM, while no cytotoxicity was observed at any lower concentrations (FIG. 7C). A 48 hour MTT assay was also performed with HCV4 and a similar toxicity profile was obtained (data not shown).

We also assessed the impact of the HCV4 peptide on NS5A-augmented IRES-mediated translation. The cells in the above-mentioned bicistronic reporter system were treated with peptide and transfected with the reporter construct and either NS5A or GFP. The peptide blocked NS5A-augmented IRES-mediated translation as shown in FIG. 5D.

### Discussion

Here we report a direct interaction between NS5A and HSP70 using purified recombinant proteins and in vitro pull-down assays. This suggests that no other viral or cellular protein is necessary for the interaction between NS5A and HSP70.

NS5A is known as a multifunctional regulator of HCV life cycle (Tellinghuisen et al. (2008) J. Virol., 82: 1073-1083; He et al. (2003) J. General Virol., 84: 535-543; Hughes et al. (2009) J. General Virol., 90: 1329-1334). NS5A domains II and III have been implicated in viral genome replication and virion assembly (Tellinghuisen et al. (2008) J. Virol., 82: 1073-1083; Hughes et al. (2009) J. General Virol., 90: 1329-1334). Furthermore, all three domains have been shown to bind viral RNA (Foster et al. (2010) J. Virol., 84: 9267-9277). None of the NS5A domains, however, have been previously ascribed to regulation of viral IRES-mediated translation. In this study, we have shown that NS5A domain I plays an important role in regulation of viral IRES-mediated translation through a beta-sheet hairpin structure. We have shown that expression of the C-terminal 34 amino acids of NS5A domain I (C34) and treatment with the C34 hairpin peptide derivative (HCV4) block NS5A-augmented IRES-mediated translation. This effect is sequence specific as peptides of equal length from NS5A domains II and III and an arginine tag control peptide (also present in HCV4 peptide) fail to produce similar results. We have previously shown that Quercetin treatment and HSP70 knock-down block NS5Aaugmented IRES-mediated translation (Gonzalez et al. (2009) Hepatology50: 1756-1764). Taken together, our data implicates the C34 hairpin in NS5A domain I in the regulation of viral IRES-mediated translation.

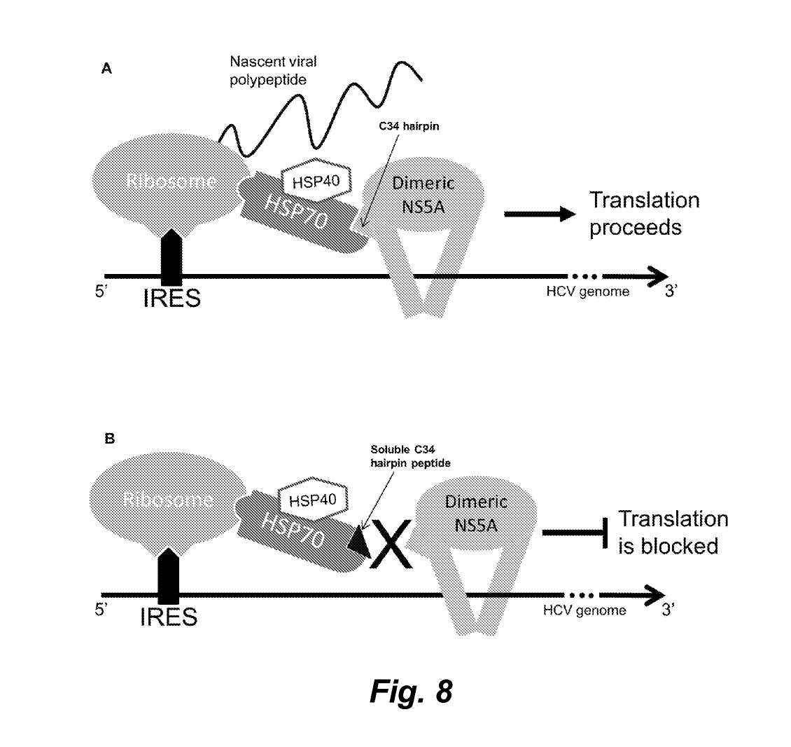

Our current model of NSSA-augmented IRES-mediated translation consists of a complex of NS5A, HSP70, HSP40, and probably additional, yet unidentified, factors such as the ribosome which may stabilize the translation complex (FIG. 8). The crystal structure of dimeric NS5A domain I predicts an RNA-binding cleft between the two claw-like domain I units (Tellinghuisen et al. (2005) Nature, 435: 374-379). It has also been reported that HSP70 (specifically the HSPA1A isoform used in this study) is able to bind to the 40S ribosomal subunit (Cornivelli et al.,(2003) Shock, 20: 320-325). Our finding that NS5A directly interacts with HSP70, therefore, provides a possible link between the NS5A dimers and the ribosome both of which interact with the RNA template. This interaction may be responsible for stabilizing the components of the translation machinery on the RNA template during the initiation and/or elongation of translation.

Deletion studies of NS5A domain I narrowed the HSP70-binding site to the C-terminus of NSSA domain I (C34). Recently it was shown that the region of NSSA encompassing amino acids 221-302 may be responsible for the NS5A/HSP70 interaction (Chen et al. (2010) J. Biol. Chem., 285: 28183-28190). This region includes most of the N terminus of NSSA domain II and some of the linker peptide between domains I and II. Flag-tagged deletion mutants within this region were shown to significantly reduce co-immunoprecipitation of HSP70 in 293T cell systems. While this region of NS5A does not directly bind HSP70, as shown by our in vitro interaction studies using purified proteins, it may represent an indirect interaction facilitated by adaptor protein(s). However, further biochemical studies are needed to verify an indirect interaction.

Without being bound to a particular theory, we believe that the hairpin in C34 is also the site of NS5A/HSP70 interaction. This is supported by the fact that the C34 hairpin is the only region of C34 with a secondary structure, based on the crystal structure of NS5A domain I (Tellinghuisen et al. (2005) Nature, 435: 374-379). It has been previously reported that HSP27 also interacts with NS5A domain I, and the site of interaction was found to be within amino acids 1-181 of NS5A domain I (Choi et al. (2004) Biochm. Biophys. Res. Comm., 318: 514-519). We note that the C34 hairpin structure lies exactly at the C terminus of this 181 amino acid region. Thus, it may be possible that, in addition to HSP70, other HSPs either interact with the C34 hairpin or are also in complex with HSP70.

HSP70 is able to non-specifically bind to a large number of hydrophobic peptide sequences (nascent or denatured peptides), which is mediated by the C-terminal substrate-binding domain (SBD) of HSP70 and allows the client-peptide to attain its native conformation (Mayer and Bukau (2005) Cellular Mol. Life. Sci., 62: 670-684). The N-terminal nucleotide binding domain (NBD) of HSP70 does not interact with these peptides directly. Rather, it binds ATP and hydrolyzes it to ADP to induce conformational changes in SBD to facilitate its function (Id.). The hydrophobicity plot of C34 shows that this region is not significantly hydrophobic. Indeed the hairpin region of C34 is hydrophilic (FIG. 6C). Our findings reveal a specific and sequence-dependent interaction between HSP70 and NS5A as there are many regions in NS5A domain I as well as domains II and III that are significantly more hydrophobic than C34 (FIG. 6C). This specific interaction is further verified by our discovery that only the HSP70-NBD interacts with NS5A directly. Thus, our data indicate that NS5A is not a substrate for HSP70-SBD. Furthermore, the HSP70-NBD is known to interact with proteins that regulate HSP70 chaperone activity. These proteins include BAGS, co-chaperones of the J-domain proteins (such as HSP40), and HSP110 all of which interact with HSP70-NBD (Mayer and Bukau (2005) Cellular Mol. Life. Sci., 62: 670-684; Arakawa et al. (2010) Structure, 18: 309-319; Polier et al. (2008) Cell, 133: 1068-1079; Vembar et al. (2009) J. Biol. Chem., 284: 32462-32471). Thus, interactions with NBD of HSP70 are an important regulatory mechanism, and it may be possible that NS5A modulates HSP70 activity as well resulting in increased IRES activity.

It is known that in the context of chronic HCV infection, a number of viral proteins including NS5A are able to dramatically alter the host gene expression and evade the innate immune response to viral infection resulting in low efficacy of the currently available pegylated interferon-α (PEG-IFN) and ribavirin treatment. Our findings demonstrate the significance of the NS5A/HSP70 interaction and the role of HSPs in HCV life cycle, in particular IRES-mediated viral protein translation. Therefore, the C34 modified hairpin peptides are good candidates for HCV therapy. Furthermore, considering the potency of the C34 hairpin peptide in suppressing viral translation levels, treatment with this peptide may significantly improve the efficacy of PEG-IFN and ribavirin treatment in patients resistant to these compounds or allow for IFN free therapy in combination with other antiviral agents, which may be beneficial for patients unable to receive IFN therapy.

### Example 2

### Comparison of HCV4 Peptide with other Antiviral Agents

Table 3 shows the IC50 of the HCV4 peptide compared to the IC⁵⁰s reported in the literature for a number of other known antiviral compounds. As shown therein, the HCV4 peptide shows higher antiviral activity than most other known antiviral compounds.

TABLE 3
Antiviral activity of HCV4 compared to the antiviral activity
reported for a number of known antiviral compounds.

| | Compound | IC₅₀ in Cell Culture | IC₅₀ in in vitro Assays |
|---|---|---|---|
| 1 | Telaprevir | 354 nM or 280 nM | 10 nM² (protease assay) |
| 2 | Boceprevir | 200 nM or 300- | |
| | | 900 nM | |
| 3 | TMC435 | | 13 nM⁴ (protease assay) |
| 4 | Bl 201335 | 3.1-50 nM (replicon)⁵ | |
| 5 | MK-7009 | 5-15 nM (replicon)⁶ | |
| | (Vaniprevir) | | |
| 6 | ITMN-191 | 1.8 nM (replicon)⁷ | |
| | (Da noprevir) | | |
| 7 | R7128 (PSI-6130) | 600 nM (replicon)⁸ | |
| 8 | GS-9190 | | >71 μM (polymerase |
| | (Tegobuvir) | | assay)⁹ |
| 9 | Filibuvir | | 50 nM (primer extension |
| 10 | GS-5885 | 34 pM and 4 pM | |
| 11 | SCY-635 | 140 nM (replicon)¹² | |
| 12 | HCV4 | 542 pM (reporter | |
| | | virus) | |

¹Summary of Product Characteristics. Available from: www.ema.europa.eu/docs/en GB/document library/EPAR - Product Information/human/002313/WC500115529.pdf.
²Highlights of Prescribing Information. Available from: www.accessdata.fda.govIdrugsatfda docs/label/2012/201917s0041 bl.pdf.
³Assessment Report. Available from: www.ema.europa.eu/docs/en GB/document library/EPAR - Public assessment report/human/002332/WC500109789.pdf.
⁴Lenz, et al. (2010) Antimicrobial Agents and Chemotherapy, 54(5): 1878-1887.
⁵White et al. (2010) Antimicrobial Agents and Chemotherapy, 54(11): 4611-4618.
⁶Liverton et al. (2010) Antimicrobial Agents and Chemotherapy, 54(1): 305-311.
⁷HCV Protease Inhibitor ITMN-191 Resistance. Available from: www.natap.org/2007/EASL/EASL 44.htm.
⁸Ma et al. (2007) J. Biol. Chem., 282(41): 29812-29820.
⁹Shih et al. (2011) Antimicrobial Agents and Chemotherapy, 55(9): 4196-4203.
¹⁰Vi et al. (2012) Antimicrobial Agents and Chemotherapy, 56(2): 830-837.
¹¹Lawitz et al. (2012) J. Hepatology, 57(1): 24-31.
¹²Preclinical Evaluation of SCY-635, a Cyclophilin Inhibitor with Potent anti-HCV Activity. Available from: www.scynexis.com/wp-content/uploads/2011/04/AASLD-2006-10-poster-934.pdf.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A peptide that inhibits intracellular production of a virus that has an internal ribosome entry site, said peptide ranging in length from 8 to 34 amino acids and comprising an amino acid sequence according to the formula
(SEQ ID NO: 1)
X⁴-X⁵-X⁶-X⁷-X⁸-X⁹-X¹⁰-X¹¹
or the retro, inverso, or retro-inverso form of said amino acid sequence wherein:
X⁴ is an amino acid selected from the group consisting of Phe, ²Nal, Dpa, and Trp, or conservative substitutions thereof;
X⁵ is an amino acid selected from the group consisting of Leu, and Arg, or conservative substitutions thereof;
X⁶ is an amino acid selected from the group consisting of Val, Chg, Cys, and Cys^{tBu}, or conservative substitutions thereof X⁷ is an amino acid selected from the group consisting of Gly, Pro, and (D)Pro, or conservative substitutions thereof;
X⁸ is an amino acid selected from the group consisting of Leu, and Cha, or conservative substitutions thereof;
X⁹ is an amino acid selected from the group consisting of Asn, and His, or conservative substitutions thereof;
X¹⁰ is an amino acid selected from the group consisting of Gln, Glu, and Arg, or conservative substitutions thereof; and
X¹¹ is an amino acid selected from the group consisting of Tyr, Bip, and Dpa, or conservative substitutions thereof.

2. The peptide of claim 1, wherein said peptide comprises an amino acid sequence according to the formula
(SEQ ID NO: 2)
X¹ₘ-X²ₙ-X³-X⁴-X⁵-X⁶-X⁷-X⁸-X⁹-X¹⁰-X¹¹-X¹²-X¹³ₒ-X¹⁴ₚ
or the retro, inverso, or retro-inverso form of said amino acid sequence wherein
m, n, o, and p are independently 0 or 1;
X¹ is present or absent and when present is Arg, or a conservative substitution thereof;
X² is present or absent and when present is an amino acid sequence selected from the group consisting of Val, and Cys, or conservative substitutions thereof;
X³ is an amino acid selected from the group consisting of Thr, Ser, and Arg, or conservative substitutions thereof;
X⁴ is an amino acid selected from the group consisting of Phe, ²Nal, Dpa, and Trp, or conservative substitutions thereof;
X⁵ is an amino acid selected from the group consisting of Leu, and Arg, or conservative substitutions thereof;
X⁶ is an amino acid selected from the group consisting of Val, Chg, Cys, and Cys^{StBu}, or conservative substitutions thereof;
X⁷ is an amino acid selected from the group consisting of Gly, Pro, and (D)Pro, or conservative substitutions thereof;
X⁸ is an amino acid selected from the group consisting of Leu, and Cha, or conservative substitutions thereof;
X⁹ is an amino acid selected from the group consisting of Asn, and His, or conservative substitutions thereof;
X¹⁰ is an amino acid selected from the group consisting of Gln, Glu, and Arg, or conservative substitutions thereof;
X¹¹ is an amino acid selected from the group consisting of Tyr, Bip, and Dpa, or conservative substitutions thereof;
X¹² is an amino acid selected from the group consisting of Leu, Pro, and Arg, or conservative substitutions thereof;
X¹³ is present or absent and when present is an amino acid selected from the group consisting of Val, and Cys, or conservative substitutions thereof; and
X¹⁴ is present or absent and, when present is an Arg or a conservative substitution thereof, or where said peptide comprise the retro form of said sequence, or where said peptide comprises the inverso form of said sequence or where said peptide comprise the retro-inverso form of said sequence.

3. (canceled)

4. The peptide of claim 3, wherein:
X⁴ is Phe or a conservative substitution thereof;
X⁶ is Val or a conservative substitution thereof;
X⁸ is Leu or a conservative substitution thereof; and
X¹⁰ is Tyr, or a conservative substitution thereof.

5. (canceled)

6. The peptide of claim 5, wherein:
X⁴ is selected from the group consisting of Dpa, ²Nal and Trp;
X⁶ is selected from the group consisting of Chg, and Cys(tBut); and
X″ is selected from the group consisting of Bip and Dpa.

7. The peptide of claim 1, wherein the amino acid sequence of said peptide comprises a sequence selected from the group consisting of Phe-Leu-Val-Gly-Leu-Asn-Gln-Tyr (SEQ ID NO:6), Phe-Arg-Val-Gly-Leu-His-Glu-Tyr (SEQ ID NO:7), Phe-Arg-Val-Gly-Leu-His-Glu-Tyr (SEQ ID NO:8), Phe-Arg-Val-Pro-Cha-His-Glu-Tyr (SEQ ID NO:9), Phe-Arg-Val-Pro-Cha-His-Arg-Tyr (SEQ ID NO:10), Phe-Arg-Val-Pro-Cha-His-Arg-Tyr (SEQ ID NO:11), Phe-Arg-Val-Pro-Cha-His-Arg-Bip (SEQ ID NO:12), Phe-Arg-Val-Pro-Cha-His-Arg-Dpa (SEQ ID NO:13), Phe-Arg-Chg-Pro-Cha-His-Arg-Tyr (SEQ ID NO:14), Phe-Arg-Cys^{tBu}-Pro-Cha-His-Arg-Tyr (SEQ ID NO:15), ²Nal-Arg-Val-Pro-Cha-His-Arg-Tyr (SEQ ID NO:16), Dpa-Arg-Val-Pro-Cha-His-Arg-Tyr (SEQ ID NO:17), Trp-Arg-Val-Pro-Cha-His-Arg-Dpa (SEQ ID NO:18), ²Nal-Arg-Chg-Pro-Cha-His-Arg-Bip (SEQ ID NO:19), ²Nal-Arg-Val-Pro-Cha-His-Arg-Tyr (SEQ ID NO:20), ²Nal-Arg-Val-Pro-Cha-His-Arg-Tyr (SEQ ID NO:21), ²Nal-Arg-Val-Pro-Cha-His-Arg-Tyr (SEQ ID NO:22), ²Nal-Arg-Val-Pro-Cha-His-Arg-Tyr (SEQ ID NO:23), ²Nal-Arg-Chg-Pro-Cha-His-Arg-Tyr (SEQ ID NO:24), ²Nal-Arg-Chg-Pro-Cha-His-Arg-Tyr (SEQ ID NO:25), Val-Thr-Phe-Leu-Val-Gly-Leu-Asn-Gln-Tyr-Leu-Val (SEQ ID NO:26), Val-Ser-Phe-Arg-Val-Gly-Leu-His-Glu-Tyr-Pro-Val (SEQ ID NO:27), Tyr-Phe-Val-Pro-His-Glu-Ser-Gly-Arg-Val-Val-Leu (SEQ ID NO:28), Cys-Ser-Phe-Arg-Val-Gly-Leu-His-Glu-Tyr-Pro-Cys (SEQ ID NO:29), Cys-Ser-Phe-Arg-Val-Pro-Cha-His-Glu-Tyr-Pro-Cys (SEQ ID NO:30), Arg-Cys-Arg-Phe-Arg-Val-Pro-Cha-His-Arg-Tyr-Arg-Cys-Arg (SEQ ID NO:31), Arg-Cys-Arg-Phe-Arg-Val-Pro-Cha-His-Arg-Tyr-Arg-Cys-Arg (SEQ ID NO:32), Arg-Cys-Arg-Phe-Arg-Val-Pro-Cha-His-Arg-Bip-Arg-Cys-Arg (SEQ ID NO:33), Arg-Cys-Arg-Phe-Arg-Val-Pro-Cha-His-Arg-Dpa-Arg-Cys-Arg (SEQ ID NO:34), Arg-Cys-Arg-Phe-Arg-Chg-Pro-Cha-His-Arg-Tyr-Arg-Cys-Arg (SEQ ID NO:35), Arg-Cys-Arg-Phe-Arg-Cys-Pro-Cha-His-Arg-Tyr-Arg-Cys-Arg (SEQ ID NO:36), Arg-Cys-Arg-²Nal-Arg-Val-Pro-Cha-His-Arg-Tyr-Arg-Cys-Arg (SEQ ID NO:37), Arg-Cys-Arg-Dpa-Arg-Val-Pro-Cha-His-Arg-Tyr-Arg-Cys-Arg (SEQ ID NO:38), Arg-Cys-Arg-Trp-Arg-Val-Pro-Cha-His-Arg-Dpa-Arg-Cys-Arg (SEQ ID NO:39), Arg-Cys-Arg-²Nal-Arg-Chg-Pro-Cha-His-Arg-Bip-Arg-Cys-Arg (SEQ ID NO:40), Cys-Arg-²Nal-Arg-Val-Pro-Cha-His-Arg-Tyr-Arg-Cys(SEQ ID NO:41), Cys-Arg-²Nal Nal-Arg-Val-Pro-Cha-His-Arg-Tyr-Arg-Cys (SEQ ID NO:42), Arg-²Nal-Arg-Val-Pro-Cha-His-Arg-Tyr-Arg-Cys (SEQ ID NO:43), Arg-Nal-Arg-Val-Pro-Cha-His-Arg-Tyr-Arg (SEQ ID NO:44), Cys-Arg-²Nal-Arg-Chg-Pro-Cha-His-Arg-Tyr-Arg-Cys (SEQ ID NO:45), and Arg-Nal-Arg-Chg-Pro-Cha-His-Arg-Tyr-Arg (SEQ ID NO:46).

8. (canceled)

9. The peptide of claim 1, wherein said peptide comprises one or more “D” amino acids.

10. The peptide of claim 9, wherein X⁷ comprises a (D)proline.

11. (canceled)

12. The peptide of claim 1, wherein the amino acid sequence of said peptide comprises the amino acid sequence of residues X²-X¹² as shown in Table 1 or FIG. 9.

13. The peptide of claim 1, wherein the amino acid sequence of said peptide comprises an amino acid sequence of a peptide shown in Table 1 or FIG. 9.

14-19. (canceled)

20. The peptide of claim 1, wherein said peptide further comprises a cell penetrating peptide attached to the amino or carboxyl terminus.

21. The peptide of claim 20, wherein said cell penetrating peptide comprises a poly Arg tag of the form R-(Ahx-R)₆, or said said cell penetrating peptide comprises the amino acid sequence of a peptide shown in Table 2.

22. (canceled)

23. The peptide of claim 1, wherein said peptide is a beta peptide.

24. The peptide of claim 1, wherein said peptide forms a beta hairpin conformation, or wherein said peptide is a cyclized peptide.

25-27. (canceled)

28. The peptide of claim 1, wherein said peptide bears a first protecting group on the carboxyl terminus and/or a second protecting group on the amino terminus.

29. The peptide of claim 28, wherein first protecting group when present, and/or said second protecting group when present is protecting group selected from the group consisting of acetyl, amide, and 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh),Tosyl (Tos), 2,2,5,7,8-pentamethyl chroman-6-sulphonyl (Pmc), 4-methylbenzyl (MeBz1), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bz1), 2-chlorobenzyloxycarbonyl (2-Cl—Z),2-bromobenzyloxycarbonyl (2-Br—Z), Benzyloxymethyl (Bom), t-butoxycarbonyl (Boc), cyclohexyloxy (cHxO),t-butoxymethyl (Bum), t-butoxy (tBuO), t-Butyl (tBu), Acetyl (Ac), and Trifluoroacetyl (TFA).

30-32. (canceled)

33. The peptide of claim 1, wherein said peptide is formulated as a pharmaceutical formulation.

34. The peptide of claim 33, wherein said peptide is formulated in a lipid or liposome, with a non-covalent carrier, or with a recombinant vault nanocapsule.

35-38. (canceled)

39. A pharmaceutical formulation comprising a peptide of claim 1 and a pharmaceutically acceptable excipient.

40-41. (canceled)

42. A method of inhibiting intracellular production of a positive sense RNA virus that has an internal ribosome entry site (IRES) said method comprising delivering to a cell containing said virus a peptide of claim 1 in an amount sufficient to reduce or block production of said virus.

43. A method of inhibiting NS5A-mediated IRES activity and viral infection, said method comprising delivering to a cell containing said virus a peptide of claim 1 in an amount sufficient to reduce or block NS5A-mediated IRES activity of said virus.

44. (canceled)

45. A method of treating a subject infected with a positive sense RNA virus that has an internal ribosome entry site (IRES) said method comprising administering to said subject a peptide of claim 1 in an amount to reduce or block propagation of said virus.

46-64. (canceled)


SEQUENCE LISTING (101 sequences)

SEQ ID NO: 1 - protein, 8 aa, Artificial
  Synthetic peptide
XXXXXXXX

SEQ ID NO: 2 - protein, 14 aa, Artificial
  Synthetic peptide
XXXXXXXXXXXXXX

SEQ ID NO: 3 - protein, 8 aa, Artificial
  Synthetic peptide
FLVGLNQY

SEQ ID NO: 4 - protein, 8 aa, Artificial
  Synthetic peptide
FRVGLHEY

SEQ ID NO: 5 - protein, 8 aa, Artificial
  Synthetic peptide
FRVGLHEY

SEQ ID NO: 6 - protein, 8 aa, Artificial
  Synthetic peptide
FRVXXHEY

SEQ ID NO: 7 - protein, 8 aa, Artificial
  Synthetic peptide
FRVXXHRY

SEQ ID NO: 8 - protein, 8 aa, Artificial
  Synthetic peptide
FRVXXHRY

SEQ ID NO: 9 - protein, 8 aa, Artificial
  Synthetic peptide
FRVXXHRX

SEQ ID NO: 10 - protein, 8 aa, Artificial
  Synthetic peptide
FRVXXHRX

SEQ ID NO: 11 - protein, 8 aa, Artificial
  Synthetic peptide
FRXXXHRY

SEQ ID NO: 12 - protein, 8 aa, Artificial
  Synthetic peptide
FRCXXHRY

SEQ ID NO: 13 - protein, 8 aa, Artificial
  Synthetic peptide
XRVXXHRY

SEQ ID NO: 14 - protein, 8 aa, Artificial
  Synthetic peptide
XRVXXHRY

SEQ ID NO: 15 - protein, 8 aa, Artificial
  Synthetic peptide
WRVXXHRX

SEQ ID NO: 16 - protein, 8 aa, Artificial
  Synthetic peptide
XRXXXHRX

SEQ ID NO: 17 - protein, 8 aa, Artificial
  Synthetic peptide
XRVXXHRY

SEQ ID NO: 18 - protein, 8 aa, Artificial
  Synthetic peptide
XRVXXHRY

SEQ ID NO: 19 - protein, 8 aa, Artificial
  Synthetic peptide
XRVXXHRY

SEQ ID NO: 20 - protein, 8 aa, Artificial
  Synthetic peptide
XRVXXHRY

SEQ ID NO: 21 - protein, 8 aa, Artificial
  Synthetic peptide
XRXXXHRY

SEQ ID NO: 22 - protein, 8 aa, Artificial
  Synthetic peptide
XRXXXHRY

SEQ ID NO: 23 - protein, 12 aa, Artificial
  Synthetic peptide
VTFLVGLNQYLV

SEQ ID NO: 24 - protein, 12 aa, Artificial
  Synthetic peptide
VSFRVGLHEYPV

SEQ ID NO: 25 - protein, 12 aa, Artificial
  Synthetic peptide
YFVPHESGRVVL

SEQ ID NO: 26 - protein, 12 aa, Artificial
  Synthetic peptide
CSFRVGLHEYPC

SEQ ID NO: 27 - protein, 12 aa, Artificial
  Synthetic peptide
CSFRVPXHEYPC

SEQ ID NO: 28 - protein, 14 aa, Artificial
  Synthetic peptide
RCRFRVXXHRYRCR

SEQ ID NO: 29 - protein, 14 aa, Artificial
  Synthetic peptide
RCRFRVXXHRYRCR

SEQ ID NO: 30 - protein, 14 aa, Artificial
  Synthetic peptide
RCRFRVXXHRXRCR

SEQ ID NO: 31 - protein, 14 aa, Artificial
  Synthetic peptide
RCRFRVXXHRXRCR

SEQ ID NO: 32 - protein, 13 aa, Artificial
  Synthetic peptide
RCRFRXXHRYRCR

SEQ ID NO: 33 - protein, 14 aa, Artificial
  Synthetic peptide
RCRFRCXXHRYRCR

SEQ ID NO: 34 - protein, 14 aa, Artificial
  Synthetic peptide
RCRXRVXXHRYRCR

SEQ ID NO: 35 - protein, 14 aa, Artificial
  Synthetic peptide
RCRXRVXXHRYRCR

SEQ ID NO: 36 - protein, 14 aa, Artificial
  Synthetic peptide
RCRWRVXXHRXRCR

SEQ ID NO: 37 - protein, 14 aa, Artificial
  Synthetic peptide
RCRXRXXXHRXRCR

SEQ ID NO: 38 - protein, 12 aa, Artificial
  Synthetic peptide
CRXRVXXHRYRC

SEQ ID NO: 39 - protein, 13 aa, Artificial
  Synthetic peptide
CRXXRVXXHRYRC

SEQ ID NO: 40 - protein, 11 aa, Artificial
  Synthetic peptide
RXRVXXHRYRC

SEQ ID NO: 41 - protein, 10 aa, Artificial
  Synthetic peptide
RXRVXXHRYR

SEQ ID NO: 42 - protein, 12 aa, Artificial
  Synthetic peptide
CRXRXXXHRYRC

SEQ ID NO: 43 - protein, 10 aa, Artificial
  Synthetic peptide
RXRXXXHRYR

SEQ ID NO: 44 - protein, 34 aa, Artificial
  Synthetic peptide
LREEVSFRVGLHEYPVGSQLPCEPEPDVAVLTSM

SEQ ID NO: 45 - protein, 9 aa, Artificial
  Synthetic peptide
FRVGLHEYP

SEQ ID NO: 46 - protein, 9 aa, Artificial
  Synthetic peptide
XXXXXXXXX

SEQ ID NO: 47 - protein, 10 aa, Artificial
  Synthetic peptide
XXXXXXXXXX

SEQ ID NO: 48 - protein, 12 aa, Artificial
  Synthetic peptide
VTFLVGLNQYLV

SEQ ID NO: 49 - protein, 12 aa, Artificial
  Synthetic peptide
VSFRVGLHEYPV

SEQ ID NO: 50 - protein, 12 aa, Artificial
  Synthetic peptide
YFVPHESGRVVL

SEQ ID NO: 51 - protein, 12 aa, Artificial
  Synthetic peptide
CSFRVGLHEYPC

SEQ ID NO: 52 - protein, 27 aa, Artificial
  Synthetic peptide
RXRXRXRXRXRXRXXCSFRVXXHEYPC

SEQ ID NO: 53 - protein, 14 aa, Artificial
  Synthetic peptide
RCRFRVXXHRYRCR

SEQ ID NO: 54 - protein, 16 aa, Artificial
  Synthetic peptide
XXRCRFRVXXHRYRCR

SEQ ID NO: 55 - protein, 14 aa, Artificial
  Synthetic peptide
RCRFRVXXHRXRCR

SEQ ID NO: 56 - protein, 14 aa, Artificial
  Synthetic peptide
RCRFRVXXHRXRCR

SEQ ID NO: 57 - protein, 14 aa, Artificial
  Synthetic peptide
RCRFRXXXHRYRCR

SEQ ID NO: 58 - protein, 14 aa, Artificial
  Synthetic peptide
RCRFRXXXHRYRCR

SEQ ID NO: 59 - protein, 14 aa, Artificial
  Synthetic peptide
RCRXRVXXHRYRCR

SEQ ID NO: 60 - protein, 14 aa, Artificial
  Synthetic peptide
RCRXRVXXHRYRCR

SEQ ID NO: 61 - protein, 14 aa, Artificial
  Synthetic peptide
RCRWRVXXHRXRCR

SEQ ID NO: 62 - protein, 14 aa, Artificial
  Synthetic peptide
RCRXRXXXHRXRCR

SEQ ID NO: 63 - protein, 12 aa, Artificial
  Synthetic peptide
CRXRVXXHRYRC

SEQ ID NO: 64 - protein, 12 aa, Artificial
  Synthetic peptide
CRXRVXXHRYRC

SEQ ID NO: 65 - protein, 11 aa, Artificial
  Synthetic peptide
RXRVXXHRYRC

SEQ ID NO: 66 - protein, 10 aa, Artificial
  Synthetic peptide
RXRVXXHRYR

SEQ ID NO: 67 - protein, 12 aa, Artificial
  Synthetic peptide
CRXRXXXHRYRC

SEQ ID NO: 68 - protein, 10 aa, Artificial
  Synthetic peptide
RXRXXXHRYR

SEQ ID NO: 69 - protein, 12 aa, Artificial
  Synthetic peptide
XXXXXXPXXXXX

SEQ ID NO: 70 - protein, 13 aa, Artificial
  Synthetic peptide
GRKKRRQRRRPPQ

SEQ ID NO: 71 - protein, 13 aa, Artificial
  Synthetic peptide
GRRRRRRRRRPPQ

SEQ ID NO: 72 - protein, 11 aa, Artificial
  Synthetic peptide
YGRKKRRQRRR

SEQ ID NO: 73 - protein, 8 aa, Artificial
  Synthetic peptide
RRRRRRRR

SEQ ID NO: 74 - protein, 6 aa, Artificial
  Synthetic peptide
RRRRRR

SEQ ID NO: 75 - protein, 18 aa, Artificial
  Synthetic peptide
LLIILRRRIRKQAHAHSK

SEQ ID NO: 76 - protein, 17 aa, Artificial
  Synthetic peptide
TRQARRNRRRRWRERQR

SEQ ID NO: 77 - protein, 8 aa, Artificial
  Synthetic peptide
RRRRRRRW

SEQ ID NO: 78 - protein, 13 aa, Artificial
  Synthetic peptide
GRKKRRQRRRPWQ

SEQ ID NO: 79 - protein, 15 aa, Artificial
  Synthetic peptide
RRRRNRTRRNRRRVR

SEQ ID NO: 80 - protein, 19 aa, Artificial
  Synthetic peptide
KMTRAQRRAAARRNRWTAR

SEQ ID NO: 81 - protein, 13 aa, Artificial
  Synthetic peptide
TRRQRTRRARRNR

SEQ ID NO: 82 - protein, 19 aa, Artificial
  Synthetic peptide
KLTRAQRRAAARKNKRNTR

SEQ ID NO: 83 - protein, 17 aa, Artificial
  Synthetic peptide
NAKTRRHERRRKLAIER

SEQ ID NO: 84 - protein, 16 aa, Artificial
  Synthetic peptide
RQIKIWFQNRRMKWKK

SEQ ID NO: 85 - protein, 16 aa, Artificial
  Synthetic peptide
RQIKIWFQNRRMKWKK

SEQ ID NO: 86 - protein, 16 aa, Artificial
  Synthetic peptide
RQIKIFFQNRRMKFKK

SEQ ID NO: 87 - protein, 18 aa, Artificial
  Synthetic peptide
KLALKLALKALKAALKLA

SEQ ID NO: 88 - protein, 16 aa, Artificial
  Synthetic peptide
RQIRIWFQNRRMRWRR

SEQ ID NO: 89 - protein, 16 aa, Artificial
  Synthetic peptide
KQIKIWFQNKKMKWKK

SEQ ID NO: 90 - protein, 22 aa, Artificial
  Synthetic peptide
MDAQTRRRERRAEKQAQWKAAN

SEQ ID NO: 91 - protein, 18 aa, Artificial
  Synthetic peptide
TAKTRYKARRAELIAERR

SEQ ID NO: 92 - protein, 16 aa, Artificial
  Synthetic peptide
TRRNKRNRIQEQLNRK

SEQ ID NO: 93 - protein, 12 aa, Artificial
  Synthetic peptide
SQMTRQARRLYV

SEQ ID NO: 94 - protein, 27 aa, Artificial
  Synthetic peptide
GWTLNSAGYLLGKINLKALAALAKKIL

SEQ ID NO: 95 - protein, 21 aa, Artificial
  Synthetic peptide
AGYLLGKINLKALAALAKKIL

SEQ ID NO: 96 - protein, 18 aa, Artificial
  Synthetic peptide
KLALKLALKALKAALKLA

SEQ ID NO: 97 - protein, 30 aa, Artificial
  Synthetic peptide
WEAKLAKALAKALAKHLAKALAKALKACEA

SEQ ID NO: 98 - protein, 20 aa, Artificial
  Synthetic peptide
GLFKALLKLLKSLWKLLLKA

SEQ ID NO: 99 - protein, 21 aa, Artificial
  Synthetic peptide
KETWWETWWTEWSQPKKKRKV

SEQ ID NO: 100 - protein, 27 aa, Artificial
  Synthetic peptide
GALFLGFLGAAGSTMGAWSQPKSKRKV

SEQ ID NO: 101 - protein, 15 aa, Artificial
  Synthetic peptide
AAVALLPAVLLALLP